(19) United States
(12) Patent Application Publication
(10) Pub. No.: US 2011/0059970 A1
(43) Pub. Date: Mar. 10, 2011

(54) 4-PHENYL-1,3-THIAZOLES AND 4-PHENYL-1,3-OXAZOLES DERIVATIVES AS CANNABINOID RECEPTOR LIGANDS

(75) Inventor: Jeremiah Harnett, Gif sur Y vette (FR)

(73) Assignee: IPSEN PHARMA S.A.S., BOULOGNE-BILLANCOURT (FR)

(21) Appl. No.: 12/678,279

(22) PCT Filed: Sep. 11, 2008
(86) PCT No.: PCT/FR08/01270

§ 371 Date: Nov. 22, 2010

(30) Foreign Application Priority Data
Sep. 13, 2007 (FR) 0706434

(51) Int. Cl. A61K 31/5377; C07D 417/04; A61K 31/427; C07D 401/14; A61K 31/454; C07D 413/14; C07D 403/14; A61K 31/496; A61P 35/00; A61P 37/08; A61P 29/00; A61P 27/02; A61P 19/10; A61P 25/00; A61P 9/00

(52) U.S. Cl. 514/236.8; 548/205; 514/365; 546/209; 514/326; 544/133; 544/367; 514/254.02

(57) ABSTRACT

The present invention relates to new 4-phenyl-1,3-azole derivatives having the general formula (I)

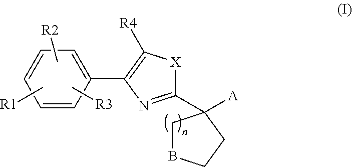

wherein R1, R2, R3, R4, X, A, B, and n are variable, in a racemic form, an enantiomeric form or any combinations thereof. These compounds exhibit affinity for cannabinoid receptors and may therefore be used as drugs to treat or prevent pathological states and diseases in which one or more of these receptors are involved. The invention also relates to pharmaceutical compositions containing said products and to the use thereof to prepare a drug.

The present application relates to new 4-phenyl-1,3-azole derivatives. These products have high affinity for certain subtypes of cannabinoid receptors, in particular the CB2 receptors. They are particularly interesting for treating pathological states and diseases in which one or more cannabinoid receptors are involved. The invention also relates to pharmaceutical compositions containing said products and to the use thereof to prepare a drug.

The cannabinoids present in Indian hemp (Cannabis sativa) are psychoactive components including close to 6 different molecules, the predominant one being delta-9-tetrahydrocannabinol. The therapeutic effect of cannabis has been known since the ancient Chinese dynasties, 5000 years ago, when cannabis was used for the treatment of asthma, migraine and gynecological disorders. Cannabis extracts were recognised and included in the American pharmacopoeia in 1850.

Cannabinoids are known to have effects on the central nervous system and/or on the cardiovascular system. These effects include memory impairment, euphoria and sedation. Cannabinoids also increase the heart rate and alter systemic blood pressure. Peripheral effects related to bronchial constriction, immunomodulation and inflammation have also been observed. More recently, it has been shown that cannabinoids modulate cellular and humoral immune responses and have anti-inflammatory properties.

However, in spite of all these properties, the therapeutic use of cannabinoids is controversial because of their psychoactive effects (cause of dependence) and also because of their multiple side effects which have not yet been fully characterised.

In addition, two cannabinoid receptors, CB1 and CB2, have been identified and cloned. CB1 is predominantly expressed in the central nervous system whereas CB2 is expressed in peripheral tissues, mainly in the immune system. These two receptors are members of the family of G protein-coupled receptors and their inhibition is related to adenylate cyclase activity.

In order to respond to manufacturers' requirements it has become necessary to find compounds that are able to modulate cannabinoid receptor activity selectively i.e. products that are selective for one particular receptor subtype.

Thus, there is considerable interest in compounds with high selective affinity for the CB2 receptor. Indeed compounds that specifically modulate the activity of CB2 receptors, either directly or indirectly, can produce clinically useful effects without binding to the CB1 receptors and therefore without affecting the central nervous system, thereby providing a rational therapeutic approach for a great variety of pathological states such as immune disorders, inflammation, osteoporosis and renal ischaemia.

The problem that the invention proposes to solve is to provide products with affinity for cannabinoid receptors and more particularly products that are selective for the CB2 receptor subtype.

The invention also proposes the use of compounds having the general formula (I) for the treatment and prevention of pathological states and diseases associated with cannabinoid receptor activity including, but not limited to, disorders of cell proliferation such as cancer, immune disorders and autoimmune diseases, allergic diseases, inflammation, pain, eye disorders, lung conditions, osteoporosis, gastrointestinal disorders, neurodegenerative diseases, cardiovascular disease. Among these diseases, the following diseases or conditions can be cited for example: immune system disorders, particularly autoimmune diseases: psoriasis, lupus erythematosus, connective tissue diseases, Sjögren's syndrome, ankylosing spondylitis, rheumatoid arthritis, reactive arthritis, undifferentiated spondyloarthropathy, Behçcet's disease, autoimmune haemolytic anaemia, multiple sclerosis, amyotrophic lateral sclerosis, amyloidosis, graft rejection, diseases affecting the plasma cell lineage; allergic diseases: delayed- or immediate-type hypersensitivity, allergic rhinitis, contact dermatitis, allergic conjunctivitis; parasitic, viral or bacterial infectious diseases: AIDS, meningitis; amyloidosis, diseases affecting the lineages of the lymphohaemopoietic system; chronic alcohol-related, viral or toxic liver disease, as well as nonalcoholic steatohepatitis and primary liver cancer; inflammatory diseases, particularly joint diseases: arthritis, rheumatoid arthritis, osteoarthritis, spondylitis, gout, vasculitis, Crohn's disease, inflammatory bowel disease and irritable bowel syndrome, pancreatitis; osteoporosis; pain: chronic inflammatory pain, neuropathic pain, acute peripheral pain; eye disorders: ocular hypertension, glaucoma; lung conditions: diseases of the airways, asthma, fibrosis, chronic bronchitis, chronic obstructive pulmonary disease, emphysema; diseases of the central nervous system and neurodegenerative disease: Tourette's syndrome, Parkinson's disease, Alzheimer's disease, senile dementia, chorea, Huntington's chorea, epilepsy, psychoses, depression, spinal cord injury; migraine, dizziness, vomiting, nausea, particularly subsequent to chemotherapy; cardiovascular disease particularly hypertension, atherosclerosis, heart attack, ischaemic heart disease; renal ischaemia; cancer: benign skin tumours, papillomas and cancerous tumours, prostate tumours, brain tumours (glioblastoma, medulloepithelioma, medulloblastoma, neuroblastoma, tumours of embryonic origin, astrocytoma, astroblastoma, ependymoma, oligodendroglioma, choroid plexus tumour, neuroepithelioma, pineal body tumour, ependymoblastoma, neuroectodermal, malignant meningioma, sarcomatosis, malignant melanoma, schwannoma); gastrointestinal diseases; obesity; diabetes.

Other advantages and characteristics of the invention will be clear from reading the description and examples given hereinbelow, which are purely illustrative and not restrictive.

Unexpectedly, the inventors demonstrated that it is possible to use compounds having the general formula (I) to modulate the activity of CB2.

The invention therefore relates to compounds having the general formula (I)

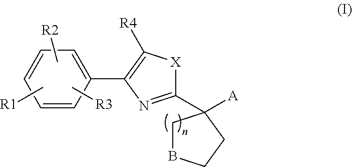

in a racemic form, an enantiomeric form or any combinations thereof, wherein:

A represents an —NR5R6, —CR5R6R7 or —OR5 group wherein R5, R6, R7 represent independently a hydrogen atom, an alkyl or cycloalkyl group;

or alternatively A represents —NR5R6, where R5 and R6 together with the nitrogen atom to which they are attached form a heterocycloalkyl;
or alternatively A represents —CR5R6R7, where R5, R6 and R7 together with the carbon atom to which they are attached form a cycloalkyl;
n represents an integer between 1 and 3 inclusive;
B represents an oxygen atom, a sulphur atom, a methylene group or an —NR9- group;
X represents an oxygen or sulphur atom;
R4 represents a hydrogen atom or an alkyl group;
R1, R2, R3 represent independently a hydrogen atom, a halogen atom, an alkyl group, —CH₂R11, —SR11, haloalkyl, —N(R9)₂, —OR10;
R9 represents a hydrogen atom or an alkyl group;
R10 represents a hydrogen atom, an alkyl or aryl group optionally substituted by one or more identical or independently different groups selected from halo, nitro, cyano or alkoxy;
R11 represents an aryl group;
or a pharmaceutically acceptable salt thereof;
it being understood that when A represents —NH₂, B does not represent a methylene group.

Unless otherwise indicated, alkyl denotes a linear or branched alkyl group consisting of 1 to 8 carbon atoms, preferably 1 to 6 carbon atoms, and more preferably 1 to 4 carbon atoms. Linear or branched alkyl with 1 to 6 carbon atoms denotes for example methyl, ethyl, propyl, isopropyl, butyl, isobutyl, sec-butyl and tent-butyl, pentyl, neopentyl, isopentyl, hexyl or isohexyl groups.

Haloalkyl denotes an alkyl group as hereinabove defined where at least one of the hydrogen atoms is substituted by a halogen atom, such as the —CF₃ group.

Unless otherwise indicated, alkoxy denotes an —O-alkyl group where the term alkyl is as hereinabove defined. Preferably, the term alkoxy represents a group such as methoxy, ethoxy, propyloxy, isopropyloxy and very preferably the methoxy group.

Unless otherwise indicated, cycloalkyl denotes a saturated carbocyclic group containing 3 to 7 carbon atoms such as cyclopropyl, cyclobutyl, cyclopentyl, cyclohexyl or cycloheptyl.

Heterocycloalkyl (or heterocyclic) as employed herein denotes a non-aromatic cyclic group containing 4 to 7 atoms, these atoms being selected from carbon, nitrogen, oxygen or sulphur, or a combination thereof such as piperidine, pyrrolidine, azetidine, morpholine, thiomorpholine, piperazine, homopiperazine groups.

In particular piperazine denotes a group

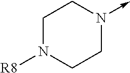

where R8 represents a hydrogen atom or an alkyl group.

In particular homopiperazine denotes a group

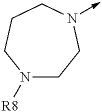

where R8 represents a hydrogen atom or an alkyl group.

Aryl denotes an unsaturated carbocyclic system containing at least one aromatic ring, preferably a group selected from phenyl, naphthyl and fluorenyl, and very preferably a phenyl group.

Preferably, the invention relates to a compound having the general formula (I) wherein R1, R2, R3 represent independently a hydrogen atom, a halogen atom, an alkyl group, —CH₂R11, —SR11, haloalkyl, —N(R9)₂, —OR10; R10 represents a hydrogen atom, an alkyl or phenyl group optionally substituted by one or more identical or independently different groups selected from halo, nitro, cyano, or alkoxy; and R11 represents a phenyl group.

Very preferably, the invention relates to a compound having the general formula (I) wherein R1, R2, R3 represent independently a hydrogen atom, a halogen atom, an alkyl group, —CH₂R11, —SR11, haloalkyl, —N(R9)₂, —OR10; R10 represents a hydrogen atom, an alkyl or phenyl group optionally substituted by one or more identical or independently different groups selected from halo, nitro, cyano or methoxy; and R11 represents a phenyl group.

Preferably, the invention relates to a compound having the general formula (I) wherein X represents the sulphur atom.

Also preferably, the invention relates to a compound having the general formula (I) wherein X represents the oxygen atom.

Preferably, the compound of the invention has R1, R2, R3 groups representing independently a hydrogen atom, a halogen atom, an alkyl group or —OR10.

Preferably, the compound of the invention has R1, R2, R3 groups that represent independently an alkyl group or —OR10, where R10 represents a hydrogen atom or an alkyl group.

Preferably, the compound of the invention has a group A that represents an —NR5R6 group where R5 and R6 represent independently a hydrogen atom or an alkyl group.

Preferably, the compound of the invention has a group A that represents —NR5R6 where R5 and R6 together with the nitrogen atom to which they are attached form a heterocycloalkyl.

Preferably, the compound of the invention has a group B that represents an oxygen or sulphur atom.

Preferably also, the compound of the invention can have the general formula (I) wherein:

A represents independently an —NR5R6 group, where R5, R6, represent independently a hydrogen atom, an alkyl or cycloalkyl group;

or alternatively A represents —NR5R6, where R5 and R6 together with the nitrogen atom to which they are attached form a heterocycloalkyl;
n represents an integer between 1 and 3 inclusive;
B represents an oxygen atom, a sulphur atom, a methylene group or an —NR9 group;
X represents an oxygen or sulphur atom;
R4 represents a hydrogen atom or an alkyl group;
R1, R2, R3 represent independently a hydrogen atom, a halogen atom, an alkyl group, —OR10;
R9 represents a hydrogen atom or an alkyl group;
R10 represents a hydrogen atom, an alkyl or aryl group optionally substituted by one or more identical or independently different groups selected from halo, nitro, cyano, or alkoxy;
or a pharmaceutically acceptable salt thereof;
it being understood that when A represents —NH₂, B does not represent a methylene group.

Preferably also, the compound of the invention can have the general formula (I) wherein:

A represents an —NR5R6 group, where R5 and R6 represent independently a hydrogen atom, an alkyl or cycloalkyl group;
or alternatively A represents —NR5R6, where R5 and R6 together with the nitrogen atom to which they are attached form a heterocycloalkyl;
n represents an integer between 1 and 3 inclusive;
B represents an oxygen or sulphur atom;
X represents an oxygen or sulphur atom;
R4 represents a hydrogen atom or an alkyl group;
R1, R2, R3 represent independently a hydrogen atom, a halogen atom, an alkyl group, —CH₂R11, —SR11, haloalkyl, —N(R9)₂, —OR10;
R10 represents a hydrogen atom, alkyl, or an aryl group optionally substituted by one or more identical or independently different groups selected from halo, nitro, cyano or alkoxy;
R11 represents an aryl group;
or a pharmaceutically acceptable salt thereof.

Preferably, the compound of the invention can have the general formula (I) wherein:

A represents an —NR5R6 group, where R5 and R6 represent independently a hydrogen atom, an alkyl or cycloalkyl group;

or alternatively A represents —NR5R6, where R5 and R6 together with the nitrogen atom to which they are attached form a heterocycloalkyl;
n represents an integer between 1 and 3 inclusive;
B represents an oxygen or sulphur atom;
X represents an oxygen or sulphur atom;
R4 represents a hydrogen atom or an alkyl group;
R1, R2, R3 represent independently a hydrogen atom, a halogen atom, an alkyl group or —OR10;
R10 represents a hydrogen atom, alkyl, or an aryl group optionally substituted by one or more identical or independently different groups selected from halo, nitro, cyano or alkoxy;
or a pharmaceutically acceptable salt thereof.

Preferably, the compound of the invention is selected from the following compounds or salts thereof:
{4-[4-(3,5-di-tert-butyl-4-methoxyphenyl)-1,3-thiazol-2-yl]tetrahydro-2H-pyran-4-yl}amine
{4-[4-(3,5-di-tert-butyl-4-methoxyphenyl)-1,3-thiazol-2-yl]tetrahydro-2H-pyran-4-yl}dimethylamine hydrochloride
4-[2-(4-aminotetrahydro-2H-pyran-4-yl)-1,3-thiazol-4-yl]-2,6-di-tert-butylphenol
2,6-di-tert-butyl-4-{2-[4-(dimethylamino)tetrahydro-2H-pyran-4-yl]-1,3-thiazol-4-yl}phenol hydrochloride
4-[2-(4-aminotetrahydro-2H-thiopyran-4-yl)-1,3-thiazol-4-yl]-2,6-di-tert-butylphenol
2,6-di-tert-butyl-4-{2-[4-(dimethylamino) tetrahydro-2H-thiopyran-4-yl]-1,3-thiazol-4-yl}phenol hydrochloride
2,6-di-tert-butyl-4-{2-[3-(dimethylamino) tetrahydrofuran-3-yl]-1,3-thiazol-4-yl}phenol hydrochloride
{4-[4-(4-tert-butylphenyl)-1,3-thiazol-2-yl]tetrahydro-2H-pyran-4-yl}dimethylamine hydrochloride
(4-{-4-[3,5-bis(trifluoromethyl)phenyl]-1,3-thiazol-2-yl}tetrahydro-2H-pyran-4-yl)dimethylamine hydrochloride
{4-[4-(3,5-di-tert-butylphenyl)-1,3-thiazol-2-yl]tetrahydro-2H-pyran-4-yl}amine
{4-[4-(3,5-di-tert-butylphenyl)-1,3-thiazol-2-yl]tetrahydro-2H-pyran-4-yl}dimethylamine hydrochloride
{4-[4-(4-methoxy-3,5-dimethylphenyl)-1,3-thiazol-2-yl]tetrahydro-2H-pyran-4-yl}dimethylamine hydrochloride
{4-[4-(3-tert-butyl-5-chloro-4-methoxyphenyl)-1,3-thiazol-2-yl]tetrahydro-2H-pyran-4-yl}dimethylamine hydrochloride
{4-[4-(3-tert-butyl-4-methoxyphenyl)-1,3-thiazol-2-yl]tetrahydro-2H-pyran-4-yl}dimethylamine hydrochloride
2,6-di-tert-butyl-4-{2-[4-(dimethylamino)tetrahydro-2H-pyran-4-yl]-1,3-oxazol-4-yl}phenol hydrochloride
N,N-dimethyl-4-[4-(4-phenoxyphenyl)-1,3-thiazol-2-yl]tetrahydro-2H-pyran-4-amine hydrochloride
2,6-di-tert-butyl-4-{2-[4-(ethylamino)tetrahydro-2H-pyran-4-yl]-1,3-thiazol-4-yl}phenol
2,6-di-tert-butyl-4-{2-[4-(diethylamino)tetrahydro-2H-pyran-4-yl]-1,3-thiazol-4-yl}phenol hydrochloride
2,6-di-tert-butyl-4-{2-[4-(methylamino)tetrahydro-2H-pyran-4-yl]-1,3-thiazol-4-yl}phenol hydrochloride
2,6-di-tert-butyl-4-[2-(4-piperidin-1-yltetrahydro-2H-pyran-4-yl)-1,3-thiazol-4-yl]phenol hydrochloride
2,6-di-tert-butyl-4-[2-(4-morpholin-4-yltetrahydro-2H-pyran-4-yl)-1,3-thiazol-4-yl]phenol
2,6-di-tert-butyl-4-{2-[4-(4-methylpiperazin-1-yl)tetrahydro-2H-pyran-4-yl]-1,3-thiazol-4-yl}phenol hydrochloride
2,6-di-tert-butyl-4-{2-[4-(dimethylamino)-1-methylpiperidin-4-yl]-1,3-thiazol-4-yl}phenol hydrochloride
2,6-di-tert-butyl-4-{2-[4-(dimethylamino)piperidin-4-yl]-1,3-thiazol-4-yl}phenol trifluoroacetate
{4-[4-(3,5-di-tert-butyl-4-methoxyphenyl)-5-methyl-1,3-thiazol-2-yl]tetrahydro-2H-pyran-4-yl}dimethylamine hydrochloride
2,6-di-tert-butyl-4-[2-(1-methoxycyclopentyl)-1,3-thiazol-4-yl]phenol
2,6-di-tert-butyl-4-[2-(1-ethylcyclopentyl)-1,3-thiazol-4-yl]phenol
(4-{4-[4-(diethylamino)phenyl]-1,3-thiazol-2-yl}tetrahydro-2H-pyran-4-yl)dimethylamine hydrochloride
{4-[4-(4-benzylphenyl)-1,3-thiazol-2-yl]tetrahydro-2H-pyran-4-yl}dimethylamine hydrochloride
2-chloro-6-(4-{2-[4-(dimethylamino)tetrahydro-2H-pyran-4-yl]-1,3-thiazol-4-yl}phenoxy)benzonitrile hydrochloride
(4-{-4-[2-chloro-4-(4-chlorophenoxy)phenyl]-1,3-thiazol-2-yl}tetrahydro-2H-pyran-4-yl)dimethylamine hydrochloride
N,N-dimethyl-4-{4-[4-(4-nitrophenoxy)phenyl]-1,3-thiazol-2-yl}tetrahydro-2H-pyran-4-amine hydrochloride
N,N-dimethyl-4-{4-[4-(phenylthio)phenyl]-1,3-thiazol-2-yl}tetrahydro-2H-pyran-4-amine hydrochloride
(4-{4-[4-(4-methoxyphenoxy)phenyl]-1,3-thiazol-2-yl}tetrahydro-2H-pyran-4-yl)dimethylamine hydrochloride
{4-[4-(3,5-di-tert-butyl-2-methoxyphenyl)-1,3-thiazol-2-yl]tetrahydro-2H-pyran-4-yl}dimethylamine hydrochloride

More preferably, the compound of the invention is selected from the following compounds or salts thereof:
{4-[4-(3,5-di-tert-butyl-4-methoxyphenyl)-1,3-thiazol-2-yl]tetrahydro-2H-pyran-4-yl}dimethylamine hydrochloride
2,6-di-tert-butyl-4-{2-[4-(dimethylamino)tetrahydro-2H-pyran-4-yl]-1,3-thiazol-4-yl}phenol hydrochloride
2,6-di-tert-butyl-4-{2-[4-(dimethylamino)tetrahydro-2H-thiopyran-4-yl]-1,3-thiazol-4-yl}phenol hydrochloride
2,6-di-tert-butyl-4-{2-[4-(dimethylamino)tetrahydro-2H-pyran-4-yl]-1,3-oxazol-4-yl}phenol hydrochloride
2,6-di-tert-butyl-4-{2-[4-(diethylamino)tetrahydro-2H-pyran-4-yl]-1,3-thiazol-4-yl}phenol hydrochloride
2,6-di-tert-butyl-4-[2-(4-piperidin-1-yltetrahydro-2H-pyran-4-yl)-1,3-thiazol-4-yl]phenol hydrochloride

The compounds of the invention can be prepared using the processes hereinbelow described. These processes are of course given for illustrative purposes only and the skilled person will be able to make modifications he considers useful, which may equally concern the reagents and the reaction techniques and conditions.

In the description of these processes, the term “room temperature” indicates a temperature between 20° C. and 25° C.; reaction yields are indicated as a molar percentage and the groups are as hereinabove defined.

The various synthetic routes are envisaged and presented here according to the nature of group A, which can be a —NR5R6 substituted nitrogen atom, a —CR5R6R7 substituted carbon atom or an —OR5 substituted oxygen atom.

1/Synthesis of the Compounds where A Represents —NR5R6:

These compounds are obtained using a method presented in schemes 1 to 4 below.

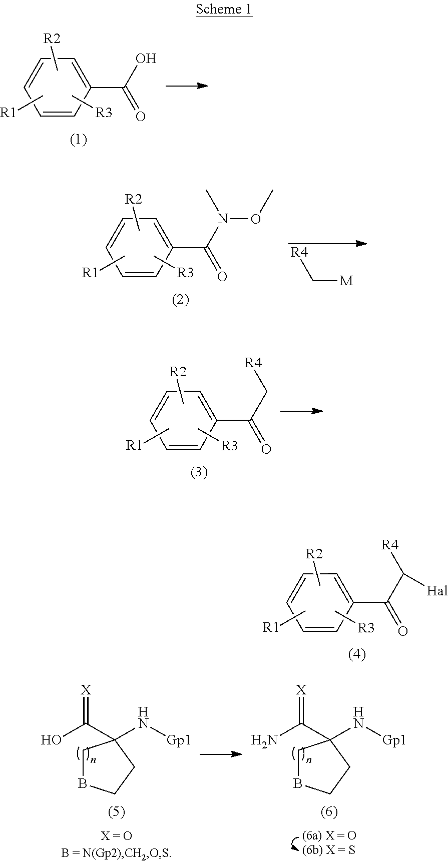

According to scheme 1, ketone derivatives (3) are converted to the corresponding α-halogenoketones of general formula (4) such as α-bromoketones by reaction with a halogenating agent such as CuBr₂ in ethyl acetate (J. Org. Chem. (1964), 29, 3459), bromine (J. Het. Chem. (1988), 25, 337) or N-bromosuccinimide (J. Amer. Chem. Soc. (1980), 102, 2838) in the presence of acetic acid in a solvent such as ethyl acetate or dichloromethane. The ketone derivatives (3) can also be converted to the corresponding α-halogenoketones of general formula (4) by reaction with other halogenating agents such as HBr or Br₂ in ether or acetic acid (Bioorg. Med. Chem. Lett. (1996), 6(3), 253-258; J. Med. Chem. (1988), 31(10), 1910-1918) or alternatively using a bromination resin (J. Macromol. Sci. Chem. (1977), A11, (3) 507-514). Ketones (3) that are not commercially available are prepared from carboxylic acids (1) converted to Weinreb carboxamides (2) by reacting carboxylic acids of general formula (1) with O,N-dimethylhydroxylamine hydrochloride in the presence of EDC and HOBT. Then the Weinreb carboxamides (2) are converted to ketones of general formula (3) by reaction with organometallic reagents R4-CH₂-M, where M is a metal group and in particular Li or MgCl or MgBr.

The compounds of general formula (6) including carboxamides (6a) and thiocarboxamides (6b) are prepared from amino acids (5) whose amine groups are protected by protecting groups Gp1 and Gp2 such as Boc, Fmoc, CBZ, Bn. The amino acids (5) are treated with HOBT ammonia (HOBT-NH₃, see experimental section) in a solvent such as DMF in the presence of BOP to form carboxamides (6a). These carboxamides (6a) react with (P₂S₅)₂ or with Lawesson's reagent in an organic solvent like dioxane or benzene at a temperature preferably between room temperature and the reflux temperature of the mixture to produce thiocarboxamides having the general formula (6b).

According to scheme 2 below, the compounds of general formula (8) are prepared from compounds of general formula (6). The conditions differ according to the nature of X. Thus, the thiazoles of general formula (8) wherein X represents S, are prepared from thiocarboxamides (6b) treated with α-halogenoketones (4) in an organic solvent such as acetone, ethanol or toluene at a temperature preferably between room temperature and the reflux temperature of the mixture to produce thiazole compounds of general formula (8). The oxazoles of general formula (8) wherein X represents O, are prepared from carboxamides (6a). These carboxamides are treated with the α-halogenoketones (4) in a polar solvent such as DMF microwave-heated to a temperature between 100° C. and 150° C. (Biotage® equipment) in a sealed tube for 45 to 90 minutes, to produce oxazoles of general formula (8).

When B represents a methylene, an oxygen atom or a sulphur atom (see scheme 2) the compounds of general formula (8) are deprotected to produce the corresponding amines having the general formula (9) using methods known to the skilled person. Gp1 may be CBZ, Boc, Fmoc or Bn. For example when Gp1 is the protecting group Fmoc, the latter is cleaved by excess of dimethylamine in an organic solvent such as tetrahydrofuran at a reaction temperature between room temperature and 55° C. The substituted amines of general formula (10, 12, 13, 14, 15) are prepared from compounds (9) using one of the following methods:
i) Condensation with corresponding aldehydes under Eschweiler-Clarke conditions (Harding, J. R.; Jones, J. R.; Lu, S-Y.; Wood, R. Tetrahedron Letters 2002, 43, 9487-9488; Torchy, S.; Barby, D. J. Chem. Res. (S) 2001, 292-293). Primary amines of general formula (9), formic acid and the corresponding aldehydes are microwave-heated in DMSO to a temperature between 130° C. and 200° C. (Biotage® equipment), in a sealed tube for 4 to 20 minutes to form secondary (10) and/or tertiary amines (12) substituted by R5′ and/or R6′ groups, it being understood that R5′ and R6′ represent independently a hydrogen atom or an alkyl group.
ii) Condensation with the corresponding aldehydes in the presence of a reducing agent (reductive amination) such as sodium triacetoxyborohydride or sodium borohydride, in a lower aliphatic alcohol such as methanol and possibly in the presence of molecular sieve, the reaction temperature being between room temperature and the reflux temperature of the mixture; reductive amination can also be achieved by microwave heating (Biotage® equipment) to form secondary (10) and/or tertiary amines (12) substituted by R5′ and/or R6′ groups, it being understood that R5′ and R6′ represent independently a hydrogen atom or an alkyl group.
iii) The amines of general formula (13) and (14) according to scheme 2 are prepared from halogenated derivatives (R5-Hal and R6-Hal where Hal is a halogen) to form secondary (13) and/or tertiary amines (14).
iv) When R5 and R6 together form a 4- to 7-membered ring, the amines of general formula (15) are prepared using the dihalogenoalkyl (11) where B2 represents NR8, NGp3, O, S or methylene. The dihalogenoalkyl (11) and compounds (9) react in a polar solvent such as ethanol in the presence of a base such as sodium bicarbonate, triethylamine, with or without sodium iodide, at a reaction temperature between room temperature and the reflux temperature of the mixture. The reaction can also be performed by microwave-heating (Biotage® equipment) to a temperature between 100° C. and 200° C. for 4 to 60 minutes to produce heterocyclic compounds having the general formula (15). When B2 represents a protected nitrogen atom NGp3, the compounds of general formula (15) are deprotected to produce the corresponding amines of general formula (16) using methods know to the skilled person; Gp3 may be CBZ, Boc, Fmoc or Bn. Next compounds (16) are alkylated using method i, ii or iii to produce compounds (17).

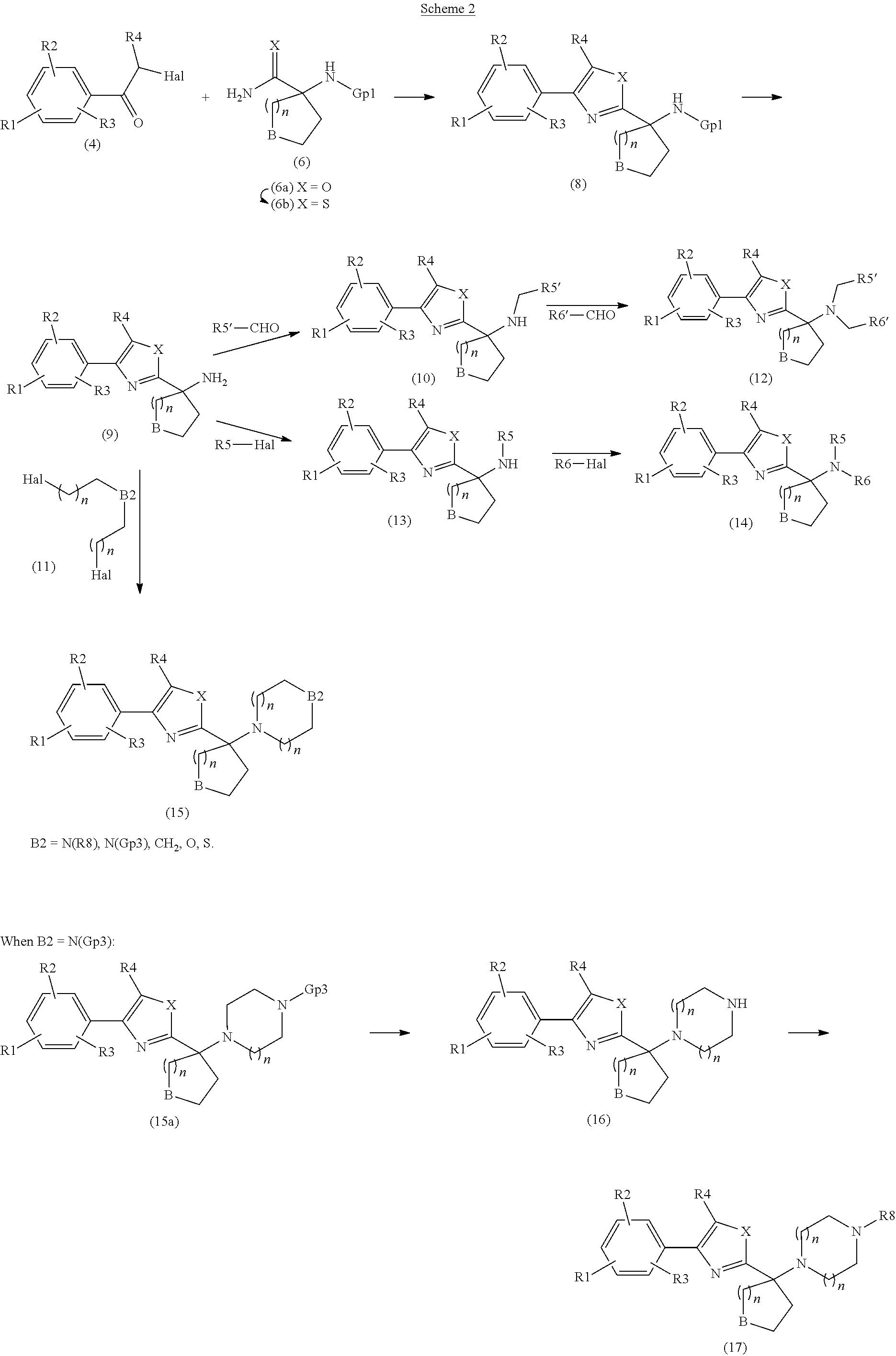

In the case of azoles (18) when B is a protected nitrogen, NGp2, the protecting groups (Gp1 and Gp2) can be selectively or nonselectively deprotected under conditions known to the skilled person, to produce compounds having the general formula (21); by route 1 if R5, R6 and R9 are identical (see scheme 3), or by routes 2 or 3 if R5, R6 and R9 are not identical (see scheme 4).

### Route 1:

In cases where A represents NR5R6, and R5, R6, and R9 are identical, Gp1 and Gp2 can be selected from CBZ, Boc, Fmoc or Bn. For example when Gp1 and Gp2 are Fmoc protecting groups, they can be deprotected by excess of dimethylamine in an organic solvent such as tetrahydrofuran, at a reaction temperature between room temperature and 55° C. When Gp1 and Gp2 are Boc protecting groups they are deprotected by bubbling HCl gas through a solvent such as ethyl acetate to produce the amines (20).

In cases where Gp1 is not identical to Gp2, the amine groups can be deprotected selectively under conditions known to the skilled person. For example when Gp1 represents a Fmoc group and Gp2 represents a Boc group, the Fmoc group is cleaved to release the amines (19) in the form of free bases under basic conditions (for example dimethylamine in THF) and the Boc group is then cleaved under acidic conditions to produce the amines (20) or vice versa. The amines (20) are then trialkylated using methods i, ii or iii to form compounds having the general formula (21).

Route 1 (R5=R6=R9)

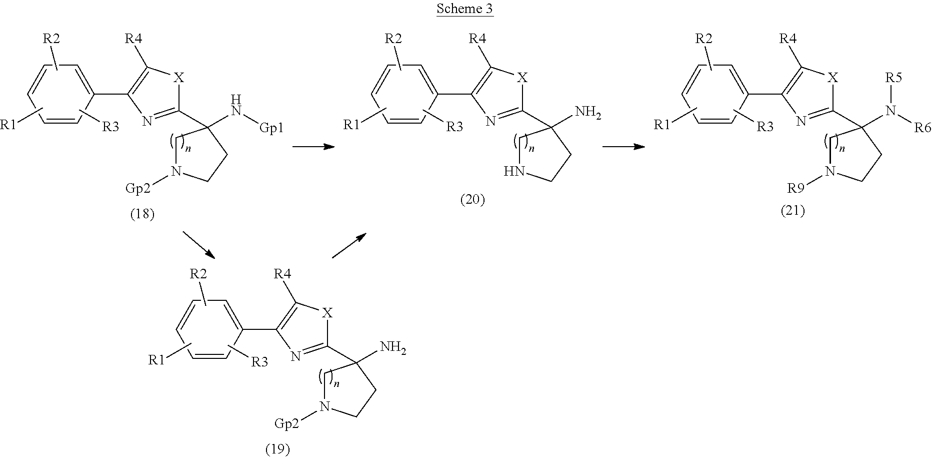

### Routes 2 and 3:

In cases where A represents NR5R6, and R5, R6 and R9 are not identical (Scheme 4), the compounds of general formula (27) are prepared according to the procedures described in scheme 4. The protecting groups Gp1 and Gp2 are selectively cleaved under conditions known to the skilled person. For example when Gp1 represents a Fmoc group and Gp2 represents a Boc group, the Fmoc group may be cleaved to release amines (19) in free base form under basic conditions (for example dimethylamine in THF) to initiate route 2, or the protecting group Gp2 may be cleaved selectively under acidic conditions to form the amines (22) in order to initiate route 3.

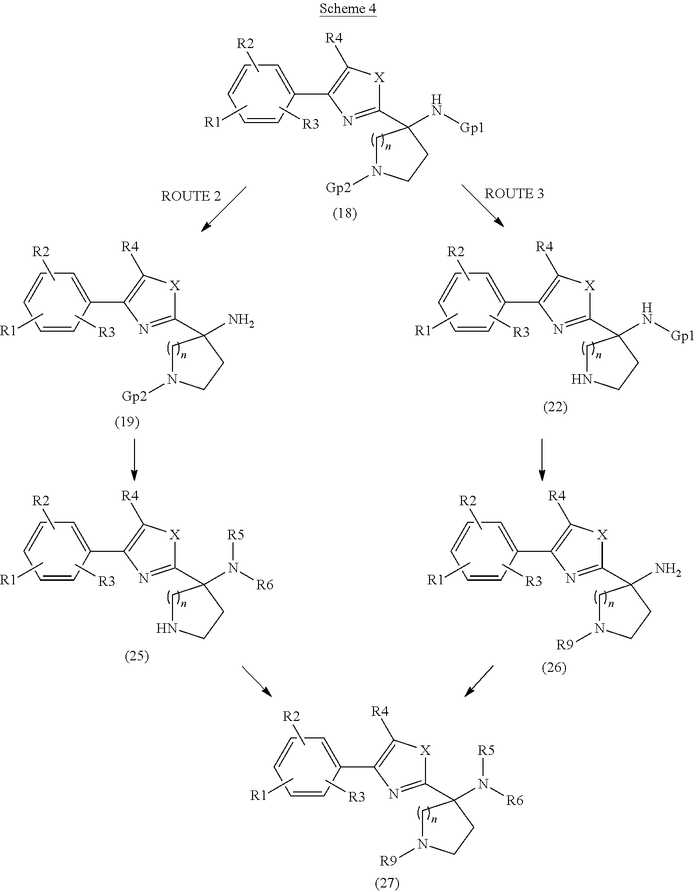

By route 2: After selective deprotection of the compounds of general formula (18), the primary amines (19) are alkylated using methods i, ii, iii or iv hereinbefore described to form the compounds (23). These compounds (23) are then deprotected to release the secondary amines (25) which are in turn alkylated using methods i, ii or iii to produce the compounds (27).

By route 3: After selective deprotection of the compounds of general formula (18) the secondary amines (22) are alkylated using methods i, ii or iii to form the compounds of general formula (24). The Gp1 group of compounds (24) is then cleaved to form primary amines (26) which are in turn alkylated using methods i, ii, iii or iv to produce the compounds (27).

2/Synthesis of the Compounds where A Represents —CR5R6R7:

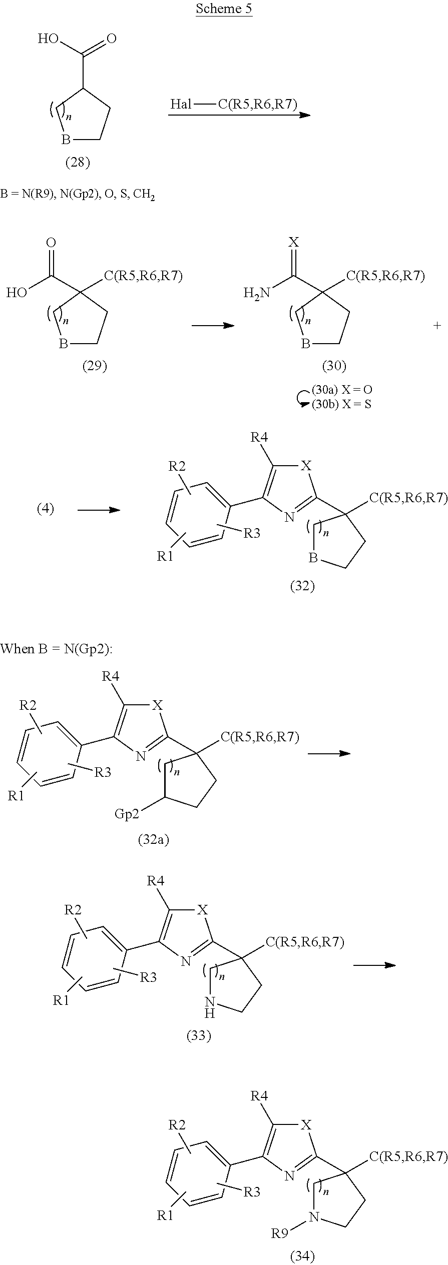

These compounds are obtained using a method summarised in scheme 5 above.

The compounds of general formula (30), including the carboxamides (30a) where X represents O and the thiocarboxamides (30b) where X represents S, are prepared from the carboxylic acids (28). The dianion of the carboxylic acids (28) can be prepared by treatment with excess of LDA (at least two eq.) at a temperature between 0° C. and −78° C. in tetrahydrofuran. This dianion is reacted with the halogenated derivatives CR5R6R7-Hal where Hal is a halogen atom to produce the compounds of general formula (29), from which the compounds of general formula (30) including the carboxamides and the thiocarboxamides (30a) and (30b) are prepared in the same way as the compounds of general formula (6) in scheme 1. The compounds of general formula (30) are reacted with the halogenated derivatives of general formula (4) in the same way as in scheme 2, providing compounds of general formula (32).

When B is a protected nitrogen atom NGp2, the amine (32a) is deprotected under classic conditions known to the skilled person, Gp2 being selected from the groups CBZ, Boc, Fmoc or Bn. For example, when Gp2 is the protecting group Fmoc, it is deprotected by excess of dimethylamine in a solvent such as tetrahydrofuran, at a reaction temperature between room temperature and 55° C., providing compounds (33). These compounds of general formula (33) can be alkylated using procedures (i) ii) or (iii) to provide the compounds of general formula (34).

3/Synthesis of the Compounds where A Represents —OR5:

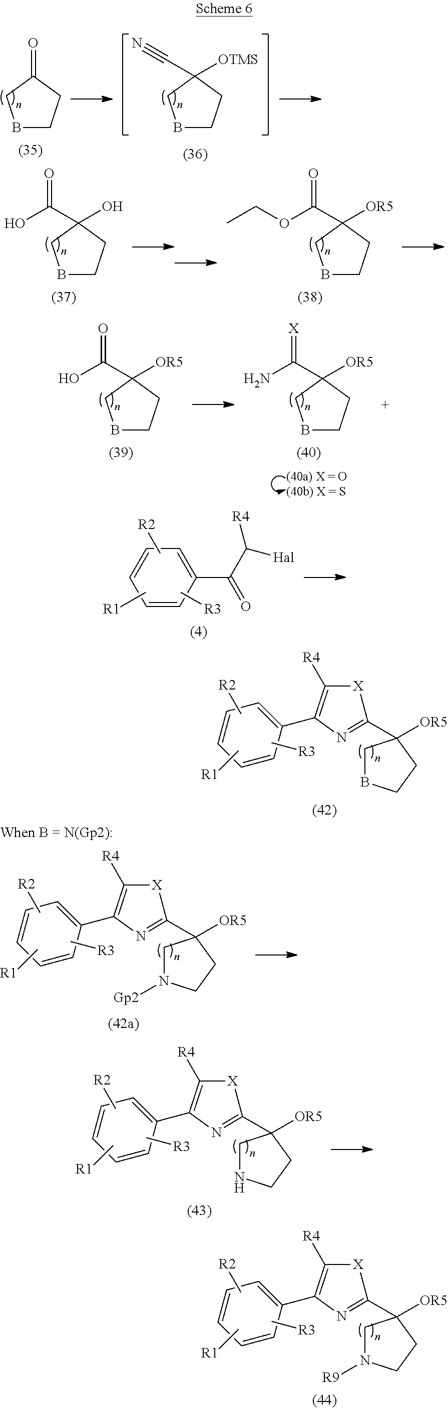

These compounds are obtained using a method summarised in scheme 6 above.

The compounds of general formula (40), including the carboxamides (40a) where X represents O and the thiocarboxamides (40b) where X represents S, are prepared from ketones of general formula (35) via cyanohydrin trimethylsilyl ethers (36). The ketones (35) react with the cyanotrimethylsilyl ether in the presence of a catalyst such as zinc iodide in an anhydrous solvent such as THF, to form the cyanohydrin trimethylsilyl ethers (36) which are not isolated but hydrolysed with HCl solution to form the hydroxy-acids (37). The acids (37) are converted to ethyl or methyl esters and the hydroxyl group is alkylated by reaction with the halogenated derivatives R5-Hal (Hal being a halogen atom) in the presence of a base such as NaH in an anhydrous solvent such as THF to form the compounds (38). The resulting esters are then saponified by the action of a base such as LiOH in tetrahydrofuran to form the acids (39) which are subsequently converted to compounds of general formula (40), including the carboxamides (40a) where X represents O and the thiocarboxamides (40b) where X represents S, using the methods hereinbefore described for the compounds of general formula (6), as illustrated in scheme 1. The compounds of general formula (40) are reacted with the halogenated derivatives of general formula (4) to produce the compounds (42) using the methods hereinbefore described for the compounds (8), as illustrated in scheme 2.

In cases where B is a protected nitrogen atom NGp2, the compounds (42a) are deprotected under classic conditions known to the skilled person to produce the amines of general formula (43) which are then alkylated according to the procedures (i) (ii) or (iii) to provide the compounds of general formula (44) using the methods hereinbefore described for the compounds (33) and (34) in scheme 5.

The present invention also relates to a process for preparing a compound having the general formula (I) as hereinabove described, characterised in that an α-halogenoketone of general formula (4)

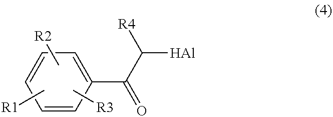

wherein R1, R2, R3 and R4 are as hereinbefore defined and Hal represents a halogen atom,
is reacted either with a compound of general formula (30)

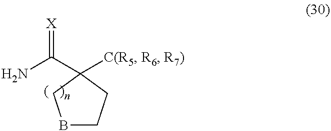

wherein X, B, R5, R6, R7 and n are as hereinbefore defined, to produce the compound of general formula (32), i.e. a compound of general formula (I) where A represents a —CR5R6R7 group,

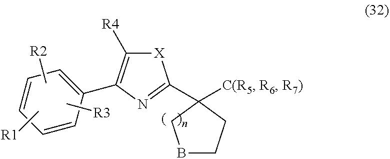

wherein R1, R2, R3, R4, X, B, n, R5, R6 and R7 are as hereinabove defined;
or is reacted with a compound of general formula (40)

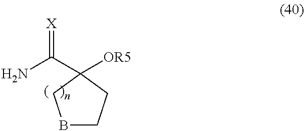

wherein X, B, R5 and n are as hereinbefore defined, to produce the compound of general formula (42) i.e. a compound of general formula (I) where A represents an —OR5 group,

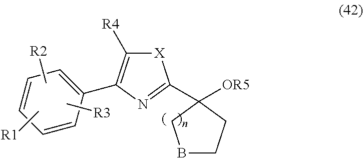

wherein R1, R2, R3, R4, X, B, n and R5 are as hereinbefore defined;
or is reacted with a compound of general formula (6)

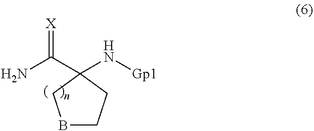

wherein X, B and n are as hereinbefore defined and Gp1 represents a protecting group, to produce the compound having the general formula (8)

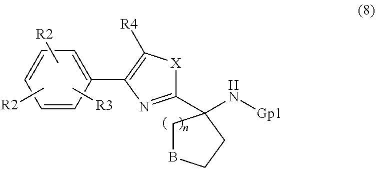

wherein R1, R2, R3, R4, X, B, n and Gp1 are as hereinbefore defined; compound of general formula (8) the protecting group of which is cleaved to produce the compound of general formula (9), i.e. a compound of general formula (I) where A represents an —NR5R6 group and R5 and R6 represent a hydrogen atom,

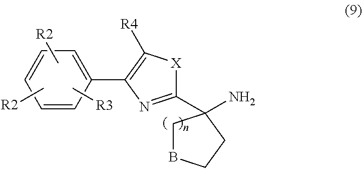

compound of general formula (I) wherein R1, R2, R3, R4, X, B, and n are as hereinbefore defined and A represents an —NR5R6 group, R5 and R6 representing a hydrogen atom, which is used as an intermediate for the synthesis of the compound of general formula (14) i.e. a compound of general formula (I) where A represents an —NR5R6 group and R5 and/or R6 do not represent a hydrogen atom,

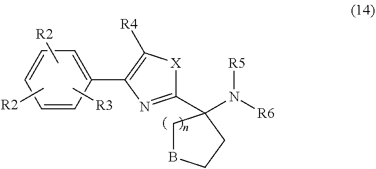

wherein R1, R2, R3, R4, X, B, n, R5 and R6 are as hereinbefore defined and R5 and/or R6 do not represent a hydrogen atom.

The present invention also relates to the compounds of general formula (I) as hereinabove described or a pharmaceutically acceptable salt thereof as a drug.

The present invention also relates to pharmaceutical compositions containing, as an active substance, a compound having the general formula (I) as hereinabove described or a pharmaceutically acceptable salt of such a compound, with at least one pharmaceutically acceptable excipient.

The present invention also relates to the use of at least one compound having the general formula (I) as hereinabove described, or a pharmaceutically acceptable salt thereof, to prepare a drug intended for the treatment or prevention of a disease or disorder selected from the following diseases or disorders: disorders of cell proliferation such as cancer, immune disorders and autoimmune diseases, allergic diseases, inflammation, pain, eye disorders, lung conditions, osteoporosis, gastrointestinal disorders, neurodegenerative diseases, cardiovascular diseases.

Preferably, the present invention also relates to the use of compounds having the general formula (I) as hereinabove described or a pharmaceutically acceptable salt thereof to prepare a drug, intended for the treatment or prevention of cancer.

Preferably, the present invention also relates to the use of at least one compound having the general formula (I) as hereinabove described, or a pharmaceutically acceptable salt thereof, to prepare a drug intended for the treatment or prevention of cancer, characterised in that the cancer to be treated or prevented is selected from cancers of the colon, rectum, stomach, lung, pancreas, kidney, testicle, breast, uterus, ovary, prostate, skin, bone, spinal cord, neck, tongue, head as well as sarcomas, carcinomas, fibroadenomas, neuroblastomas, leukaemias and melanomas.

In addition, some of the compounds of general formula (I) can be in an enantiomeric form. The present invention includes the two enantiomeric forms and any combinations thereof, including “R,S” racemic mixtures. For the sake of simplicity, when no specific configuration is indicated in the structural formulae, it should be taken to mean that both enantiomeric forms and mixtures thereof are represented.

The compound of general formula (I) or the salt thereof used according to the invention or the combination of the invention may be in the form of a solid, for example powders, granules, tablets, capsules, liposomes or suppositories. Suitable solid bases can be for example, calcium phosphate, magnesium stearate, talc, sugars, lactose, dextrin, starch, gelatin, cellulose, methylcellulose, sodium carboxymethylcellulose, polyvinylpyrrolidine and wax.

The compound of general formula (I) or the salt thereof used according to the invention or the combination of the invention may also exist in liquid form, for example solutions, emulsions, suspensions or syrups. Suitable liquid bases may be, for example, water, organic solvents such as glycerol or glycols, or blends thereof, in varying proportions, in water.

The compound of general formula (I) or its salt used according to the invention or the combination according to the invention can be administered topically, orally, parenterally, by intramuscular injection, by subcutaneous injection etc.

The anticipated dose of a product according to the present invention for the treatment of the diseases or disorders mentioned hereinabove varies according to the method of administration, the age and the body weight of the subject to be treated as well as the subject's condition, and in the end will be decided by the treating doctor or veterinarian. Such a quantity determined by the treating doctor or veterinarian is referred to herein as the “therapeutically effective quantity”.

As an indication only, the envisaged dose of a drug according to the invention is between 0.1 mg and 10 g depending on the type of active compound used.

The following examples illustrate the invention without limiting its scope.

### EXAMPLES

The melting points were determined using a Büchi B-545 capillary instrument or a Leica VMHB Kofler system.

The compounds are characterised by their molecular (MH+) peak determined by mass spectrometry (MS), using a single quadrupole mass spectrometer (Micromass, Platform model) equipped with an electrospray source, used with a resolution of 0.8 Da (50% valley definition).

### Example 1

### {4-[4-(3,5-di-tert-butyl-4-methoxyphenyl)-1,3-thiazol-2-yl]tetrahydro-2H-pyran-4-yl}amine

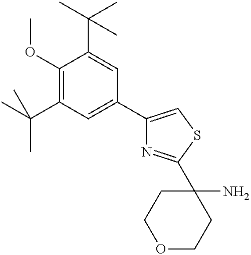

### 1.1 1-(3,5-di-tert-butyl-4-methoxyphenyl)ethanone

To a solution of 3,5-di-tert-butyl-4-hydroxy acetophenone (1.24 g; 5.0 mmol) in anhydrous tetrahydrofuran (30 ml) at 0° C. under argon, sodium hydride is added in small portions (0.240 g, 6.10 mmol) and the mixture is allowed to stand at room temperature. Methyl iodide (0.47 ml, 7.5 mmol) is added dropwise while stirring at room temperature, then refluxed for 8 hours. A water/THF mixture is added dropwise. The extraction is performed with ethyl acetate and after separation the organic phase is washed with saturated aqueous sodium chloride solution, dried over magnesium sulphate then evaporated under vacuum. The solvent is evaporated then the product is purified by silica column chromatography (eluent: ethyl acetate-heptane: 95-5). A purplish oil is obtained with a yield of 76%.

### MH+=263.4

N.B.: Potassium carbonate can be used instead of sodium hydride and DMF as the solvent to provide intermediates of the 1.1 type.

### 1.2 2-bromo-1-(3,5-di-tert-butyl-4-methoxyphenyl)ethanone

To a solution of the intermediate 1.1 (1 g; 3.81 mmol) in ethyl acetate (30 ml), copper bromide (CuBr₂) is added and the mixture refluxed for 5 hours. It is then filtered through a layer of celite and washed with ethyl acetate. The solvents are evaporated to give a brown oil that crystallises; the product is used directly in the next step without isolation.

### MH+=341.2

### 1.3 9H-fluoren-9-ylmethyl[4-(aminocarbonyl)tetrahydro-2H-pyran-4-yl]carbamate

4-{[(9H-fluoren-9-ylmethoxy)carbonyl]amino}tetrahydro-2H-pyran-4-carboxylic acid (0.5 g, 1.365 mmol) is dissolved in DMF and the reagent HOBT-NH₃* (0.253 g, 1.633 mmol) is added, followed by BOP (0.602 g, 1.36 mmol) and diisopropylethylamine (0.7 ml, 4.083 mmol) and the mixture is stirred for 12 hours. The resulting product is then extracted with ethyl acetate and water, and after separation the organic phase is washed with saturated aqueous sodium bicarbonate solution and with saturated aqueous sodium chloride solution, dried over magnesium sulphate then filtered. The filtrate is concentrated to dryness to produce a white solid, and the solid is triturated with diethyl ether then filtered. The solid is washed with ether to provide the intermediate 1.3 with a yield of 68%. The product is used directly in the next step without isolation.

Melting point: 154° C., MH+=367.3.

*Method for preparing the reagent HOBT-NH₃ used to synthesise intermediate 1.3: HOBT (20 g, 0.148 mol) is dissolved in methanol (100 ml), ammonium hydroxide is added (12 ml, NH₄OH containing 28% ammonia) and the mixture is stirred at room temperature for 12 hours. Three quarters of the volume of solvent are removed by evaporation and the solid that precipitates from it is then filtered. The solid is washed with diisopropyl ether then dried to produce a white solid with a quantitative yield.

### 1.4 9H-fluoren-9-ylmethyl[4-(aminocarbonothioyl)tetrahydro-2H-pyran-4-yl]carbamate

1.91 g (5.213 mmol) of the intermediate 1.3 is dissolved in dimethoxyethane (30 ml). Sodium bicarbonate (1.752 g; 20.85 mmol) is added, then (P₂S₅)₂ (4.634 g; 10.43 mmol) is added in small portions. The reaction medium is stirred for 24 hours. The mixture is cooled to 0° C. then saturated aqueous sodium bicarbonate solution is added and the solution is extracted with diethyl ether. Next the organic phase is dried over magnesium sulphate, filtered and concentrated under vacuum to produce a white solid with a yield of 90%.

### MH+=383.2.

### 1.5 9H-fluoren-9-ylmethyl {4-[4-(3,5-di-tert-butyl-4-methoxyphenyl)-1,3-thiazol-2-yl]tetrahydro-2H-pyran-4-yl}carbamate

The intermediate 1.4 (0.5 g, 1.3 mmol) and the intermediate 1.2 (0.446 g; 1.3 mmol) are dissolved in acetone (20 ml) under an argon atmosphere, the mixture is then heated to 50° C. for 5 hours and then stirred at room temperature for 12 hours. Next it is resuspended in saturated aqueous sodium bicarbonate solution and extracted with ethyl acetate. The organic phase is washed with saturated aqueous sodium chloride solution. The organic phase is separated, dried over magnesium sulphate, filtered and concentrated under vacuum then purified by silica column chromatography (eluent: gradient of ethyl acetate in heptane: ethyl acetate from 10% to 30%). A pale yellow foam is obtained with a yield of 77%.

### MH+=625.43

### 1.6 {4-[4-(3,5-di-tert-butyl-4-methoxyphenyl)-1,3-thiazol-2-yl]tetrahydro-2H-pyran-4-yl}amine (Example 1)

The intermediate 1.5 (0.6 g, 0.96 mmol) is dissolved in tetrahydrofuran (20 ml). Diethylamine (0.5 ml, 4.8 mmol) is added and the reaction mixture is firstly heated to reflux and then stirred at room temperature for 12 hours. It is extracted with ethyl acetate and the organic phase is washed with saturated aqueous sodium chloride solution. The organic phase is separated, dried over magnesium sulphate, filtered and concentrated under vacuum then purified by silica column chromatography (eluent: ethyl acetate-heptane: 50-50). A white solid is obtained with a yield of 52%.

Melting point: 128.4-129.7° C. MH+=403.3

### Example 2

### {4-[4-(3,5-di-tert-butyl-4-methoxyphenyl)-1,3-thiazol-2-yl]tetrahydro-2H-pyran-4-yl}dimethylamine hydrochloride

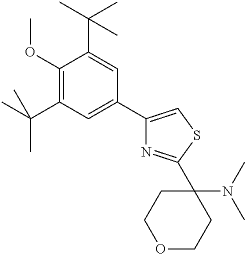

The product 1.6 in example 1 (0.180 g, 0.447 mmol) is solubilised in DMSO (2 ml) and to this is added formaldehyde (72 μl, 0.894 mmol) then formic acid (47 μl, 0.894 mmol). The reaction mixture is microwave-heated (Biotage® equipment) at 190° C. for 10 minutes. It is diluted with saturated aqueous sodium bicarbonate solution and extracted with ethyl acetate. The organic phase is then washed with saturated aqueous sodium chloride solution. Next it is dried over magnesium sulphate. It is filtered and then evaporated. Next it is purified by silica column chromatography (eluent: ethyl acetate-heptane: 50-50). A colourless oil is obtained that is directly converted to the salt without isolation. The amine is dissolved in ether (5 ml) and to this is added HCl (0.6 ml, 1M in ether), and the resulting solid is filtered and washed with diethyl ether then with isopentane and then dried under vacuum. A white solid is obtained with a yield of 38%.

Melting point: 215.0-216.7° C. MH+=431.29

### Example 3

### 4-[2-(4-aminotetrahydro-2H-pyran-4-yl)-1,3-thiazol-4-yl]-2,6-di-tert-butylphenol

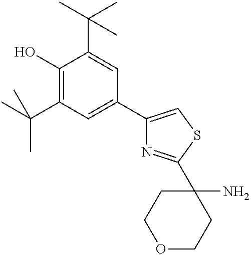

### 3.1 9H-fluoren-9-ylmethyl {4-[4-(3,5-di-tert-butyl-4-hydroxyphenyl)-1,3-thiazol-2-yl]tetrahydro-2H-pyran-4-yl}carbamate

The experimental protocol used is the same as the one described for intermediate 1.5 with commercially available 3,5-di-tert-butyl-4-hydroxy phenacyl bromide replacing the intermediate 1.2. The intermediate 3.1 is obtained in the form of a white foam, after purification by silica column chromatography (eluent: gradient of 10% to 40% ethyl acetate in heptane) with a yield of 67.3%.

### MH+=611.3

### 3.2 4-[2-(4-aminotetrahydro-2H-pyran-4-yl)-1,3-thiazol-4-yl]-2,6-di-tert-butylphenol (Example 3)

The experimental protocol used is the same as the one described for example 1 with intermediate 3.1 replacing intermediate 1.5. Example 3 is obtained in the form of a white solid after purification by silica column chromatography (eluent: gradient of 10% to 40% ethyl acetate in heptane) with a yield of 81.7%.

Melting point: 123.0-126.5° C. MH+=389.3

### Example 4

### 2,6-di-tert-butyl-4-{2-[4-(dimethylamino)tetrahydro-2H-pyran-4-yl]-1,3-thiazol-4-yl}phenol hydrochloride

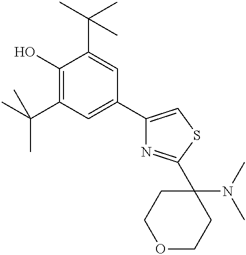

The experimental protocol used is the same as the one described for example 2, with example 3 replacing example 1. Example 4 is obtained in the form of a white solid with a yield of 86.8%.

Melting point: 255.0-257.7° C. MH+=417.3

### Example 5

### 4-[2-(4-aminotetrahydro-2H-thiopyran-4-yl)-1,3-thiazol-4-yl]-2,6-di-tert-butylphenol

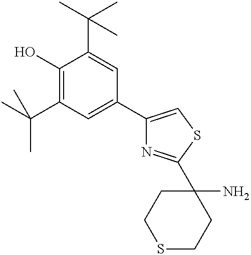

The experimental protocol used is the same as the one described for example 1, except that the synthesis starts at step 1.3 with 4-{[(9H-fluoren-9-ylmethoxy)carbonyl]amino}tetrahydro-2H-thiopyran-4-carboxylic acid replacing the 4-{[(9H-fluoren-9-ylmethoxy)carbonyl]amino}tetrahydro-2H-pyran-4-carboxylic acid and commercially available 3,5-di-tert-butyl-4-hydroxy phenacyl bromide replacing intermediate 1.2 in step 1.5. The deprotection experimental protocol described for example 1 (step 1.6) is applied and example 5 is obtained in the form of a yellow solid.

Melting point: 148.8-150.8° C. MH+=405.20

### Example 6

### 2,6-di-tert-butyl-4-{2-[4-(dimethylamino)tetrahydro-2H-thiopyran-4-yl]-1,3-thiazol-4-yl}phenol hydrochloride

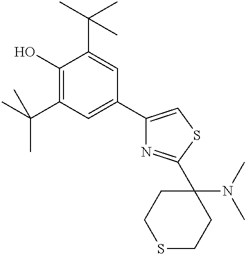

The experimental protocol used is the same as the one described for example 2, with example 5 replacing example 1. Example 6 is obtained in the form of a white solid.

Melting point: 247.2-250.0° C. MH+=433.2

### Example 7

### 2,6-di-tert-butyl-4-{2-[3-(dimethylamino)tetrahydrofuran-3-yl]-1,3-thiazol-4-yl}phenol hydrochloride

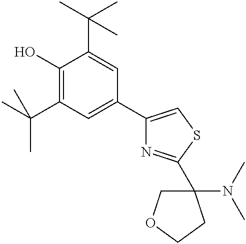

The experimental protocol used is the same as the one described for examples 1 and 2, the synthesis starting from step 1.3, 3-{(9H-fluoren-9-ylmethoxy)carbonylamino}tetrahydrofuran-3-carboxylic acid replacing the 4-{(9H-fluoren-9-ylmethoxy)carbonylamino}tetrahydro-2H-pyran-4-carboxylic acid in step 1.3 and commercially available 3,5-di-tert-butyl-4-hydroxy phenacyl bromide replacing intermediate 1.2 in step 1.5. The expected final product is obtained in the form of a white solid.

Melting point: 239.0-231.0° C. MH+=403.2

### Example 8

### {4-[4-(4-tert-butylphenyl)-1,3-thiazol-2-yl]tetrahydro-2H-pyran-4-yl}dimethylamine hydrochloride

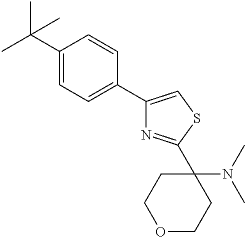

The experimental protocol used is the same as the one described for examples 1 and 2, with synthesis starting from step 1.3 and commercially available 4-tert-butyl phenacyl chloride replacing intermediate 1.2 in step 1.5. The expected final product is obtained in the form of a yellowish solid.

Melting point: 205.0-207.0° C. MH+=345.36

### Example 9

### (4-{4-[3,5-bis(trifluoromethyl)phenyl]-1,3-thiazol-2-yl}tetrahydro-2H-pyran-4-yl)dimethylamine hydrochloride

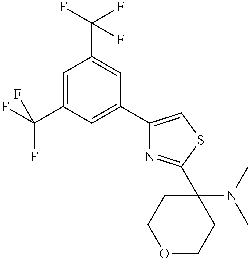

The experimental protocol used is the same as the one described for examples 1 and 2, with synthesis starting at step 1.3 and commercially available 3,5-a(trifluoromethyl)phenacyl bromide replacing intermediate 1.2 in step 1.5. The expected final product is obtained in the form of a white solid.

Melting point: 232.2-233.0° C. MH+=425.2

### Example 10

### {4-[4-(3,5-di-tert-butylphenyl)-1,3-thiazol-2-yl]tetrahydro-2H-pyran-4-yl}amine

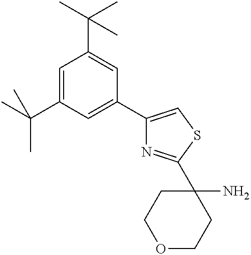

### 10.1 3,5-di-tert-butyl-N-methoxy-N-methylbenzamide

To a solution of 3,5-di-tert-butylbenzoic acid (10 g; 42.7 mmol) in anhydrous DMF (30 ml) under argon is added triethylamine (15 ml). The mixture is stirred at room temperature. After a few minutes, O,N-dimethylhydroxylamine hydrochloride (4.55 g, 46.9 mmol) is added followed by EDC (quantity) and HOBT (quantity) and the mixture is stirred at room temperature for 24 hours. It is then poured onto iced water and extracted with ethyl acetate. After separation the organic phase is washed with 10% aqueous sodium bicarbonate solution followed by saturated aqueous sodium chloride solution, dried over magnesium sulphate then evaporated under vacuum. A pale yellow oil is obtained with a quantitative yield; the product is used directly in the next step without isolation.

### MH+=278.2

### 10.2 1-(3,5-di-tert-butylphenyl)ethanone

The intermediate 10.1 (11.83 g, 42.7 mmol) dissolved in anhydrous THF (150 ml) is cooled to −30° C. then methyllithium (42.7 ml, 1.6M in ether) is added dropwise and the reaction mixture is stirred while the temperature is raised to −10° C. for 3 hours. Saturated aqueous ammonium chloride solution is poured onto the reaction mixture, the organic phase is extracted with ethyl acetate and after separation it is washed with saturated aqueous sodium chloride solution, dried over magnesium sulphate then evaporated under vacuum. A pale yellow oil is obtained which becomes orange-coloured over time, with a quantitative yield. The product is used directly in the next step without isolation.

### MH+=233.2

### 10.3 2-bromo-1-(3,5-di-tert-butylphenyl)ethanone

The experimental protocol used is the same as the one described for the intermediate 1.2 with intermediate 10.2 replacing intermediate 1.1. Intermediate 10.3 is obtained in the form of a light brown oil following purification by silica column chromatography (eluent: ethyl acetate-heptane: 5-95) with a yield of 63%.

### MH+=277.6

### 10.4 9H-fluoren-9-ylmethyl{4-[4-(3,5-di-tert-butylphenyl)-1,3-thiazol-2-yl]tetrahydro-2H-pyran-4-yl}carbamate

The experimental protocol used is the same as the one described for intermediate 1.5 with intermediate 10.3 replacing intermediate 1.4. Intermediate 10.4 is obtained in the form of a white foam following purification by silica column chromatography (eluent: ethyl acetate-heptane: 20-80) with a yield of 73%.

### MH+=595.4

### 10.5 {4-[4-(3,5-di-tert-butylphenyl)-1,3-thiazol-2-yl]tetrahydro-2H-pyran-4-yl}amine (Example 10)

The experimental protocol used is the same as the one described for example 1 (step 1.6) with intermediate 10.4 replacing intermediate 1.5. Example 10 is obtained in the form of a white crystalline solid following purification by silica column chromatography (eluent: ethyl acetate-heptane: 30-70) with a quantitative yield.

Melting point: 114.0-114.4° C. MH+=373.3

### Example 11

### {4-[4-(3,5-di-tert-butylphenyl)-1,3-thiazol-2-yl]tetrahydro-2H-pyran-4-yl}dimethylamine hydrochloride

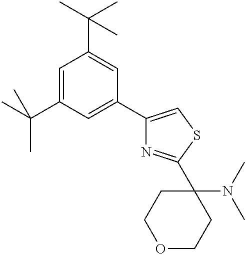

The experimental protocol used is the same as the one described for example 2 with the amine example 10 replacing example 1. Example 11 is obtained in the form of a pale yellow solid with a yield of 14%.

### MH+=401.3

### Example 12

### {4-[4-(4-methoxy-3,5-dimethylphenyl)-1,3-thiazol-2-yl]tetrahydro-2H-pyran-4-yl}dimethylamine hydrochloride

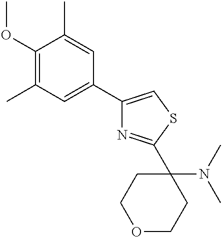

The experimental protocol used is the same as the one described for examples 1 and 2 with 3,5-dimethyl-4-hydroxyacetophenone replacing 3,5-di-tert-butyl-4-hydroxy acetophenone. The expected final product is obtained in the form of a white solid.

Melting point: 237.5-239.0° C. MH+=347.36

### Example 13

### {4-[4-(3-tert-butyl-5-chloro-4-methoxyphenyl)-1,3-thiazol-2-yl]tetrahydro-2H-pyran-4-yl}dimethylamine hydrochloride

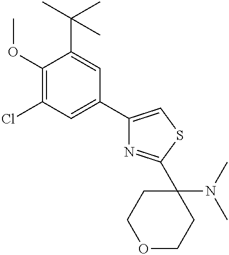

The experimental protocol used is the same as the one described for examples 1 and 2 with 1-(3-tert-Butyl-5-chloro-4-methoxyphenyl)ethanone (the method of synthesis for which is described in patent US2006/0058370 A1) replacing 3,5-di-tert-butyl-4-hydroxy acetophenone. The expected final product is obtained in the form of a white solid.

Melting point: 219.7-222.2° C. MH+=409.27

### Example 14

### {4-[4-(3-tert-butyl-4-methoxyphenyl)-1,3-thiazol-2-yl]tetrahydro-2H-pyran-4-yl}dimethylamine hydrochloride

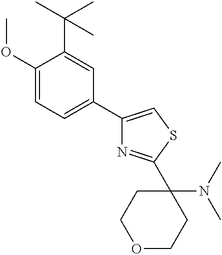

The experimental protocol used is the same as the one described for examples 1 and 2 with 1-(3-tert-Butyl-4-methoxyphenyl)ethanone (the method of synthesis for which is described in patent US2006/0058370 A1) replacing 3,5-di-tert-butyl-4-hydroxy acetophenone. The expected final product is obtained in the form of a white solid.

Melting point: 232.7-233.5° C. MH+=375.35

### Example 15

### 2,6-di-tert-butyl-4-{2-[4-(dimethylamino)tetrahydro-2H-pyran-4-yl]-1,3-oxazol-4-yl}phenol hydrochloride

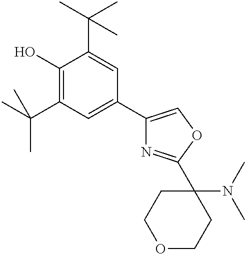

### 15.1 9H-fluoren-9-ylmethyl {4-[4-(3,5-di-tert-butyl-4-hydroxyphenyl)-1,3-oxazol-2-yl]tetrahydro-2H-pyran-4-yl}carbamate

3,5-di-tert-butyl-4-hydroxy phenacyl bromide (327 mg, 1 mmol) and the intermediate 1.4 are dissolved in anhydrous DMF (2 ml) in a Biotage® reaction tube, a spatula-tip of molecular sieve (approximately 40 mg) is added and the tube is sealed and microwave-heated with magnetic stirring at 150° C. for 1 hour and 30 minutes. The mixture is diluted with water and after separation the organic phase is washed with saturated aqueous sodium chloride solution and dried over magnesium sulphate, then filtered and evaporated under vacuum. A pale brown oil is obtained, which is purified by silica column chromatography (eluent: ethyl acetate-heptane: 20-80) and a yellowish-brown solid is obtained with a yield of 12%.

### 15.2 4-[2-(4-aminotetrahydro-2H-pyran-4-yl)-1,3-oxazol-4-yl]-2,6-di-tert-butylphenol

The experimental protocol used is the same as the one described for example 1, with intermediate 15.1 replacing intermediate 1.5. Intermediate 15.2 is obtained in the form of a brown oil following purification by silica column chromatography (eluent: chloroform-ethanol: 97-3) with a quantitative yield.

### MH+=373.32

### 15.3 2,6-di-tert-butyl-4-{2-[4-(dimethylamino)tetrahydro-2H-pyran-4-yl]-1,3-oxazol-4-yl}phenol hydrochloride (Example 15)

The experimental protocol used is the same as the one described for example 2, with intermediate 15.2 replacing example 1. Example 15 is obtained in the form of a white solid with a yield of 12%.

Melting point: 233.0-235.0° C. MH+=401.33

### Example 16

### N,N-dimethyl-4-[4-(4-phenoxyphenyl)-1,3-thiazol-2-yl]tetrahydro-2H-pyran-4-amine hydrochloride

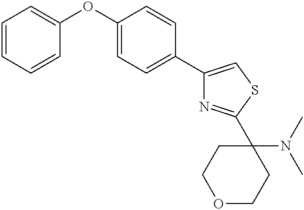

### 16.1 2-bromo-1-(4-phenoxyphenyl)ethanone

2-bromo-1-(4-phenoxyphenyl)ethanone is prepared according to the method described hereinbefore for intermediate 1.2, with 4-phenoxyacetophenone replacing intermediate 1.1. α-bromoketone is obtained in the form of a red oil, and used directly in the following step without isolation.

### 16.2 N,N-dimethyl-4-[4-(4-phenoxyphenyl)-1,3-thiazol-2-yl]tetrahydro-2H-pyran-4-amine hydrochloride (Example 16)

For the preparation of example 16, the experimental protocol used is the same as the one described for examples 1 and 2, with synthesis starting at step 1.5 and α-bromoketone 16.1 replacing intermediate 1.2. The expected final product is obtained in the form of a white solid.

Melting point: 213.0-223.2° C. MH+=381.32

### Example 17

### 2,6-di-tert-butyl-4-{2-[4-(ethylamino)tetrahydro-2H-pyran-4-yl]-1,3-thiazol-4-yl}phenol

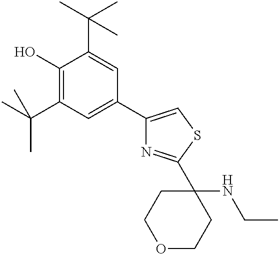

For the preparation of example 17, the experimental protocol used is the same as the one described for example 2 with example 3 replacing example 1 and acetaldehyde replacing formaldehyde. The reaction mixture is microwave-heated (Biotage® equipment) at 140° C. for 4 minutes. The expected product is obtained in the free base form and is a pale yellow solid.

### MH+=417.3

### Example 18

### 2,6-di-tert-butyl-4-{2-[4-(diethylamino)tetrahydro-2H-pyran-4-yl]-1,3-thiazol-4-yl}phenol hydrochloride

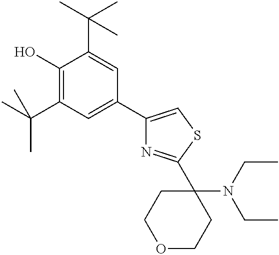

For the preparation of example 18 the experimental protocol used is the same as the one described for example 2, with example 3 replacing example 1 and acetaldehyde replacing formaldehyde (a large excess of acetaldehyde is used, about 20 eq.). The reaction mixture is microwave-heated (Biotage® equipment) at 140° C. for 16 minutes. The expected product is obtained as a hydrochloride and is a white solid.

Melting point: 208.0-211.5° C. MH+=445.3

### Example 19

### 2,6-di-tert-butyl-4-{2-[4-(methylamino)tetrahydro-2H-pyran-4-yl]-1,3-thiazol-4-yl}phenol hydrochloride

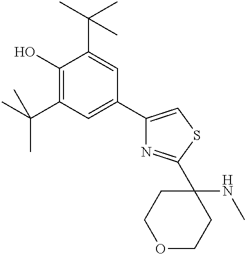

Example 3 (50 mg, 0.13 mmol) and paraformaldehyde (5 mg, 0.17 mmol) are dissolved in methanol (1 ml) in a Biotage® reaction tube, then molecular sieve (50 mg) is added followed by NaBH₄ (10 mg, 0.26 mmol). The tube is sealed with a cap and microwave-heated (Biotage®) with magnetic stirring at 120° C. for 6 minutes. The mixture is diluted in water then extracted with ethyl acetate. After separation, the organic phase is washed with saturated aqueous sodium chloride solution, dried over magnesium sulphate, filtered and then evaporated under vacuum. The residue is purified by silica column chromatography (eluent: 10% acetone in dichloromethane) and directly converted to the salt without isolation. The secondary amine (0.010 g, 0.025 mmol) is dissolved in ether (2 ml), to which HCl (0.1 ml, 1 M in ether) is then added. The resulting solid is filtered and washed with diethyl ether then dried under vacuum. A white solid is obtained with a yield of 21.1%.

Melting point: 250.0-252.0° C. MH+=403.3

### Example 20

### 2,6-di-tert-butyl-4-[2-(4-piperidin-1-yltetrahydro-2H-pyran-4-yl)-1,3-thiazol-4-yl]phenol hydrochloride

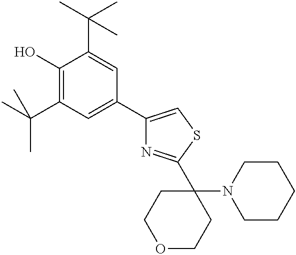

Example 3 (50 mg, 0.13 mmol) and 1,5-dibromopentane (201.4 mg, 0.876 mmol) are dissolved in ethanol (1.5 ml) in a Biotage® reaction tube. Sodium bicarbonate (38 mg, 0.450 mmol) is added then the tube is sealed with a cap and microwave-heated using a Biotage® system at 150° C. for 15 minutes. The mixture is diluted in water then extracted with ethyl acetate. After separation, the organic phase is washed with saturated aqueous sodium chloride solution, dried over magnesium sulphate, filtered and then evaporated under vacuum. The residue is purified by silica column chromatography (eluent: 20% ethyl acetate in heptane) and a colourless oil (0.03 g, 0.066 mmol) is obtained which is dissolved in ether (3 ml) to which is then added HCl (0.3 ml, 1 M in ether). The resulting solid is filtered then washed with ether and dried under vacuum. A white solid is obtained with a yield of 31.6%.

Melting point: 230.0-231.8° C. MH+=457.4.

### Example 21

### 2,6-di-tert-butyl-4-[2-(4-morpholin-4-yltetrahydro-2H-pyran-4-yl)-1,3-thiazol-4-yl]phenol

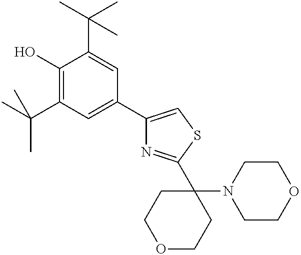

For the preparation of example 21, the experimental protocol used is the same as the one described for example 20, with bis-(2-bromoethylether) (12 eq.) replacing 1,5-dibromopentane in the presence of excess of sodium bicarbonate (14 eq.). The reaction mixture is microwave-heated (Biotage® equipment) at 160° C. for 45 minutes. The expected product is obtained in base form as a white solid with a yield of 68.8%.

Melting point: 172.0-174.0° C. MH+=459.3

### Example 22

### 2,6-di-tert-butyl-4-{2-[4-(4-methylpiperazin-1-yl)tetrahydro-2H-pyran-4-yl]-1,3-thiazol-4-yl}phenol hydrochloride

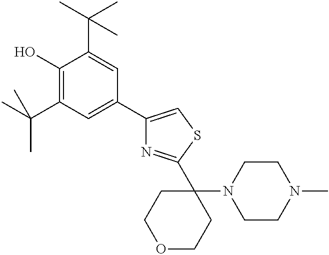

For the preparation of example 22, the experimental protocol used is the same as the one described for example 20, with mechlorethamine (6 eq.) replacing 1,5-dibromopentane. Excess of sodium bicarbonate is used (14 eq.), along with triethylamine (6 eq.) and sodium iodide (12 eq.). The reaction mixture is microwave-heated (Biotage® equipment) at 155° C. for 60 minutes. After treatment and purification by silica column chromatography (eluent: 2% ethanol in dichloromethane), the product is directly converted to the salt as in example 20 and the expected product is obtained in the form of a white solid with a yield of 68.8%.

### MH+=472.4

### Example 23

### 2,6-di-tert-butyl-4-{2-[4-(dimethylamino)-1-methylpiperidin-4-yl]-1,3-thiazol-4-yl}phenol hydrochloride

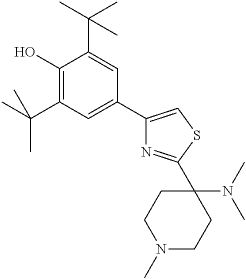

### 23.1 9H-fluoren-9-ylmethyl 4-(aminocarbonyl)-4-[(tent-butoxycarbonyl)amino]piperidine-1-carboxylate

The experimental protocol used is the same as the one described for example 1 (step 1.3), with 4-[(tert-butoxycarbonyl)amino]-1-[(9H-fluoren-9-ylmethoxy)carbonyl]piperidine-4-carboxylic acid replacing 4-{[(9H-fluoren-9-ylmethoxy)carbonyl]amino}tetrahydro-2H-pyran-4-carboxylic acid. The product 23.1 is obtained in the form of a white solid with a quantitative yield, and is used directly in the following step without isolation.

Melting point: 130° C. MH+=466.3

### 23.2 9H-fluoren-9-ylmethyl 4-(aminocarbonothioyl)-4-[(tert-butoxycarbonyl)amino]piperidine-1-carboxylate

The experimental protocol used is the same as the one described for example 1 (step 1.4), with intermediate 23.1 replacing intermediate 1.3. The product 23.2 is obtained in the form of a white solid with a yield of 30%.

### MH+=482.3

### 23.3 9H-fluoren-9-ylmethyl 4-[(tent-butoxycarbonyl)amino]-4-[4-(3,5-di-tert-butyl-4-hydroxyphenyl)-1,3-thiazol-2-yl]piperidine-1-carboxylate

The experimental protocol used is the same as the one described for example 1 (step 1.5), with intermediate 23.2 replacing intermediate 1.4. The intermediate 23.3 is obtained in the form of a white foam with a yield of 65%.

### MH+=710.5

### 23.4 9H-fluoren-9-ylmethyl 4-amino-4-[4-(3,5-di-tert-butyl-4-hydroxyphenyl)-1,3-thiazol-2-yl]piperidine-1-carboxylate

The intermediate 23.3 (0.710 g, 1 mmol) is dissolved in ethyl acetate (20 ml) through which HCl gas is bubbled for 10 minutes. The reaction mixture is concentrated to dryness, resuspended in water and extracted with ethyl acetate. After separation, the organic phase is washed with saturated aqueous sodium bicarbonate solution and saturated aqueous sodium chloride solution, dried over magnesium sulphate, filtered and then evaporated under vacuum. The residue is purified by silica column chromatography (eluent: 50% ethyl acetate in heptane) and a white foam is obtained with a yield of 94%.

### MH+=610.4

### 23.5 4-[2-(4-aminopiperidin-4-yl)-1,3-thiazol-4-yl]-2,6-di-tert-butylphenol

The experimental protocol used is the same as the one described for example 1 (step 1.6), with intermediate 23.4 replacing intermediate 1.5. The product 23.5 is obtained in the form of a light brown paste with a yield of 66%.

### MH+=388.3

### 23.6 2,6-di-tert-butyl-4-{2-[4-(dimethylamino)-1-methylpiperidin-4-yl]-1,3-thiazol-4-yl}phenol hydrochloride (Example 23)

The experimental protocol used is the same as the one described for example 2, with intermediate 23.5 replacing example 1. Example 23 is obtained in the form of a beige solid with a yield of 41%.

Melting point: 236.0-238.0° C. MH+=430.3

### Example 24

### 2,6-di-tert-butyl-4-{2-[4-(dimethylamino)piperidin-4-yl]-1,3-thiazol-4-yl}phenol trifluoroacetate

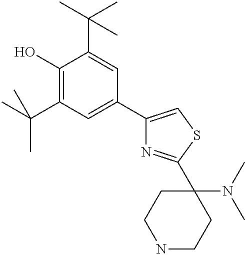

Intermediate 23.4 (280 mg, 0.459 mmol) and formaldehyde (32 mg, 1.066 mmol) are dissolved in methanol (15 ml) in the presence of molecular sieve (50 mg) and the mixture is refluxed for 5 hours then stirred at room temperature for 12 hours. NaBH₄ (20 mg, 1.066 mmol) is added in small portions then the reaction mixture is refluxed again for 5 hours (the product obtained is the di-alkylated, deprotected product). The mixture is diluted with water and extracted with ethyl acetate. After separation the organic phase is washed with saturated aqueous sodium chloride solution, dried over magnesium sulphate, filtered and then concentrated to dryness. The residue is purified by preparative reverse phase HPLC on C18 bonded silica (gradient: water+TFA 0.2 M-acetonitrile=80-20 to 60-40); an oil is obtained, with a yield of 4%.

### MH+=416.3

### Example 25

### {4-[4-(3,5-di-tert-butyl-4-methoxyphenyl)-5-methyl-1,3-thiazol-2-yl]tetrahydro-2H-pyran-4-yl}dimethylamine hydrochloride

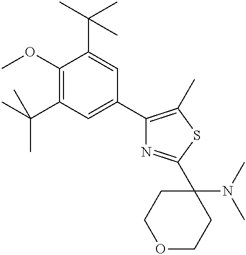

The experimental protocol used is the same as the one described for examples 1 and 2, with 1-(3,5 di-tert-Butyl-4-hydroxyphenyl)propanone (Russ. J. Org. Chem.; 33 (10) 1409-1416, 1997) replacing 3,5-di-tert-butyl-4-hydroxy acetophenone in step 1.1. The expected final product is obtained in the form of a white solid.

### MH+=445.3

### Example 26

### 2,6-di-tert-butyl-4-[2-(1-methoxycyclopentyl)-1,3-thiazol-4-yl]phenol

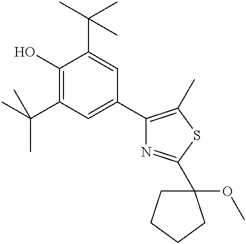

### 26.1 Methyl 1-methoxycyclopentanecarboxylate

The experimental protocol used is the same as the one described for example 1, with 1-cyclopentanol-1-methylcarboxylate ester replacing 3,5-di-tert-butyl-4-hydroxy acetophenone. Intermediate 26.1 is obtained in the form of a yellow oil with a yield of 54%.

### 26.2 1-Methoxycyclopentanecarboxylic acid

The ester 26.1 (5.85 g, 0.0367 mol) is dissolved in THF (50 ml) then an aqueous 1N LiOH solution (55 ml, 0.055 mol) is added dropwise. The mixture is stirred for 12 hours at room temperature then poured onto iced water and acidified with sufficient 1N aqueous HCl solution until the pH is adjusted to 1, then extracted with ethyl acetate. After separation the organic phase is washed with saturated aqueous sodium chloride solution, dried over magnesium sulphate, filtered and then evaporated under vacuum to form the product which is a pale yellow oil with a yield of 73%.

### 26.3 1-methoxycyclopentanecarboxamide

The experimental protocol used is the same as the one described for intermediate 1.3, with intermediate 26.2 replacing 4-{[(9H-fluoren-9-ylmethoxy)carbonyl]amino}tetrahydro-2H-pyran-4-carboxylic acid. The reaction mixture is microwave-heated (Biotage® equipment) at 60° C. for 30 minutes. After treatment the product is used directly in the following step without isolation.

### MH+=287.1

### 26.4 1-methoxycyclopentanecarbothioamide

The experimental protocol used is the same as the one described for intermediate 1.4, with intermediate 26.3 replacing intermediate 1.3. The product is obtained in the form of a white flocculent solid with a yield of 77%.

Melting point: 140° C., MH+=320.3

### 26.5 2,6-di-tert-butyl-4-[2-(1-methoxycyclopentyl)-1,3-thiazol-4-yl]phenol (Example 26)

The experimental protocol used is the same as the one described for intermediate 1.5, with intermediate 26.4 replacing intermediate 1.4; example 26 is obtained in the form of a white solid with a yield of 21%. Melting point: 121.1-122.4° C.

### Example 27

### 2,6-di-tert-butyl-4-[2-(1-ethylcyclopentyl)-1,3-thiazol-4-yl]phenol

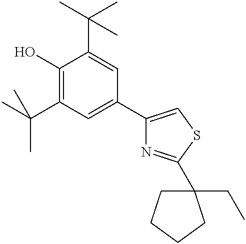

### 27.1 1-ethylcyclopentanecarboxylic acid

Diisopropylethylamine (5.874 g, 0.058 mol) is dissolved in anhydrous THF (100 ml) at 0° C. then a 1.6M solution of BuLi in heptane (33 ml) is added dropwise and stirred for 15 minutes. Cyclopentanecarboxylic acid (2.738 g, 0.024 mol) in anhydrous THF is added at 0° C. then stirred for 1 hour. Iodoethane (3.928 g, 0.0252 mol) is added at −55° C. then the temperature of the mixture is allowed to rise to −10° C. for 12 hours. The reaction is cooled to −55° C. and the reaction mixture is diluted with aqueous HCl solution (1N), extracted with ether. After separation the organic phase is washed with saturated aqueous sodium chloride solution, dried over magnesium sulphate, filtered and then evaporated under vacuum. A red oil is obtained, with a yield of 92%. The product is used directly in the following step without isolation.

### 27.2 1-ethylcyclopentanecarboxamide

The experimental protocol used is the same as the one described for intermediate 1.3, with intermediate 27.1 replacing the 4-{[(9H-fluoren-9-ylmethoxy)carbonyl]amino}tetrahydro-2H-pyran-4-carboxylic acid. Intermediate 27.2 is obtained in the form of a beige solid with a yield of 77.4%.

Melting point: 95° C.

### 27.3 1-ethylcyclopentanecarbothioamide

The experimental protocol used is the same as the one described for intermediate 1.4, with intermediate 27.2 replacing intermediate 1.3. The product is obtained in the form of pearly white solid flakes with a yield of 13%.

Melting point: 130° C.

### 27.4 2,6-di-tert-butyl-4-[2-(1-ethylcyclopentyl)-1,3-thiazol-4-yl]phenol (Example 27)

The experimental protocol used is the same as the one described for intermediate 1.5, with intermediate 27.3 replacing intermediate 1.4. Example 27 is obtained in the form of a yellow oil with a quantitative yield.

### MH+=386.3.

### Example 28

### (4-{4-[4-(diethylamino)phenyl]-1,3-thiazol-2-yl}tetrahydro-2H-pyran-4-yl)dimethylamine hydrochloride

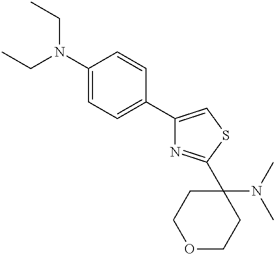

The experimental protocol used is the same as the one described for examples 1 and 2, with synthesis starting at step 1.3 and commercially available α-bromo-4-(diethylamino) acetophenone replacing intermediate 1.2 in step 1.5. The expected final product is obtained in the form of a brown solid-foam.

### MH+=360.26.

### Example 29

### {4-[4-(4-benzylphenyl)-1,3-thiazol-2-yl]tetrahydro-2H-pyran-4-yl}dimethylamine hydrochloride

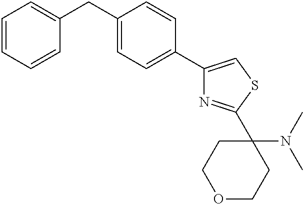

### 29.1 1-(4-benzylphenyl)-2-bromoethanone

1-(4-benzylphenyl)-2-bromoethanone is prepared according to the method described for intermediate 1.2, with (4-acetylphenyl)phenylmethane replacing intermediate 1.1. 1-(4-benzylphenyl)-2-bromoethanone is obtained in the form of a brown oil with a quantitative yield and is used directly in the following step without isolation.

### 29.2 {4-[4-(4-benzylphenyl)-1,3-thiazol-2-yl]tetrahydro-2H-pyran-4-yl}dimethylamine hydrochloride (Example 29)

For the preparation of example 29, the experimental protocol used is the same as the one described for examples 1 and 2, with synthesis starting at step 1.5 and 1-(4-benzylphenyl)-2-bromoethanone replacing intermediate 1.2 in step 1.5. The expected final product is obtained in the form of a white solid.

Melting point: 228.4-229.5° C. MH+=379.20.

### Example 30

### 2-chloro-6-(4-{2-[4-(dimethylamino)tetrahydro-2H-pyran-4-yl]-1,3-thiazol-4-yl}phenoxy)benzonitrile hydrochloride

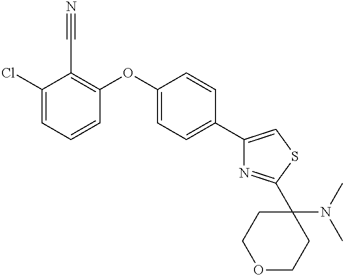

### 30.1 2-[4-(bromoacetyl)phenoxy]-6-chlorobenzonitrile

2-[4-(bromoacetyl)phenoxy]-6-chlorobenzonitrile is prepared according to the method described for intermediate 1.2, with 2-(4-acetylphenoxy)-6-chlorobenzene carbonitrile replacing intermediate 1.1. 2-[4-(bromoacetyl)phenoxy]-6-chlorobenzonitrile is obtained in the form of a grey solid by crystallisation in ether, with a quantitative yield.

Melting point: 86° C.

### 30.2 2-chloro-6-(4-{2-[4-(dimethylamino)tetrahydro-2H-pyran-4-yl]-1,3-thiazol-4-yl}phenoxy)benzonitrile hydrochloride (Example 30)

For the preparation of example 30 the experimental protocol used is the same as the one described for examples 1 and 2, with synthesis starting at step 1.5 and 2-[4-(bromoacetyl)phenoxy]-6-chlorobenzonitrile replacing intermediate 1.2 in step 1.5. The expected final product is obtained in the form of a yellow solid.

Melting point: 127-130° C. MH+=440.03.

### Example 31

### (4-{4-[2-chloro-4-(4-chlorophenoxy)phenyl]-1,3-thiazol-2-yl}tetrahydro-2H-pyran-4-yl)dimethylamine hydrochloride

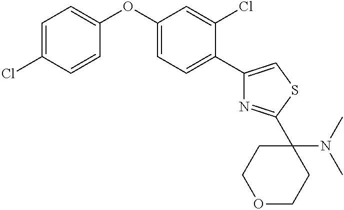

### 31.1 2-bromo-1-[2-chloro-4-(4-chlorophenoxy)phenyl]ethanone

2-bromo-1-[2-chloro-4-(4-chlorophenoxy)phenyl]ethanone is prepared according to the method described for intermediate 1.2, with 1-[2-chloro-4-(4 chlorophenoxy)phenyl]ethanone-1-one replacing intermediate 1.1. 2-bromo-1-[2-chloro-4-(4-chlorophenoxy)phenyl]ethanone is obtained in the form of yellow oil with a quantitative yield, and is used directly in the following step without isolation.

### 31.2 (4-{4-[2-chloro-4-(4-chlorophenoxy)phenyl]-1,3-thiazol-2-yl}tetrahydro-2H-pyran-4-yl)dimethylamine hydrochloride (Example 31)

For the preparation of example 31 the experimental protocol used is the same as the one described for examples 1 and 2, with synthesis starting at step 1.5 and 2-bromo-1-[2-chloro-4-(4-chlorophenoxy)phenyl]ethanone replacing intermediate 1.2 in step 1.5. The expected final product is obtained in the form of a white solid.

Melting point: 226.7-228.0° C.

### Example 32

### N,N-dimethyl-4-{4-[4-(4-nitrophenoxy)phenyl]-1,3-thiazol-2-yl}tetrahydro-2H-pyran-4-amine hydrochloride

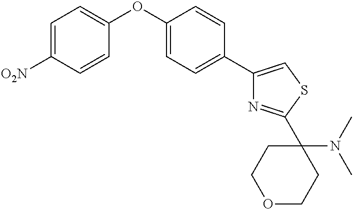

### 32.1 2-bromo-1-[4-(4-nitrophenoxy)phenyl]ethanone

2-bromo-1-[4-(4-nitrophenoxy)phenyl]ethanone is prepared according to the method described for intermediate 1.2, with 4-acetyl-4′-nitrodiphenyl ether replacing intermediate 1.1. 2-bromo-1-[4-(4-nitrophenoxy)phenyl]ethanone is obtained in the form of a pale yellow solid with a yield of 53%.

Melting point: 94° C.

### 32.2 N,N-dimethyl-4-{4-[4-(4-nitrophenoxy)phenyl]-1,3-thiazol-2-yl}tetrahydro-2H-pyran-4-amine hydrochloride (Example 32)

For the preparation of example 32 the experimental protocol used is the same as the one described for examples 1 and 2, with synthesis starting at step 1.5 and 2-bromo-1-[4-(4-nitrophenoxy)phenyl]ethanone replacing intermediate 1.2 in step 1.5. The expected final product is obtained in the form of a pale yellow solid.

Melting point: 167-169° C. MH+=426.16.

### Example 33

### N,N-dimethyl-4-{4-[4-(phenylthio)phenyl]-1,3-thiazol-2-yl}tetrahydro-2H-pyran-4-amine hydrochloride

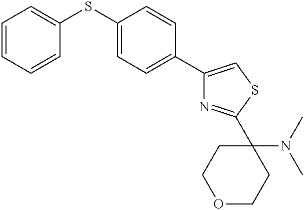

### 33.1 2-bromo-1-[4-(phenylthio)phenyl]ethanone

2-bromo-1-[4-(phenylthio)phenyl]ethanone is prepared according to the method described for intermediate 1.2 with 4-acetyldiphenylsulfide replacing intermediate 1.1. 2-bromo-1-[4-(phenylthio)phenyl]ethanone is obtained in the form of a yellow oil following purification by silica column chromatography (eluent: ethyl acetate-heptane: 20-80) with a yield of 71%.

### 33.2 N,N-dimethyl-4-{4-[4-(phenylthio)phenyl]-1,3-thiazol-2-yl}tetrahydro-2H-pyran-4-amine hydrochloride (Example 33)

For the preparation of example 33 the experimental protocol used is the same as the one described for examples 1 and 2, with synthesis starting at step 1.5 and 2-bromo-1-[4-(phenylthio)phenyl]ethanone replacing intermediate 1.2 in step 1.5. The expected final product is obtained in the form of a pale yellow solid.

Melting point: 215.0-218.8° C. MH+=397.18.

### Example 34

### (4-{4-[4-(4-methoxyphenoxy)phenyl]-1,3-thiazol-2-yl}tetrahydro-2H-pyran-4-yl)dimethylamine hydrochloride

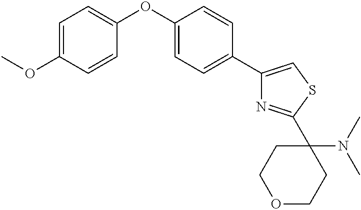

### 34.1 2-bromo-1-[4-(4-methoxyphenoxy)phenyl]ethanone

2-bromo-1-[4-(4-methoxyphenoxy)phenyl]ethanone is prepared according to the method described for intermediate 1.2, with 1-[4-(4-methoxyphenoxy)phenyl]ethan-1-one replacing intermediate 1.1. 2-bromo-1-[4-(4-methoxyphenoxy)phenyl]ethanone is obtained in the form of a brown oil that slowly crystallises, with a yield of 56%.

Melting point: 58° C.

### 34.2 (4-{4-[4-(4-methoxyphenoxy)phenyl]-1,3-thiazol-2-yl}tetrahydro-2H-pyran-4-yl)dimethylamine hydrochloride (Example 34)

For the preparation of example 34 the experimental protocol used is the same as the one described for examples 1 and 2, with synthesis starting at step 1.5 and 2-bromo-1-[4-(4-methoxyphenoxy)phenyl]ethanone replacing intermediate 1.2 in step 1.5. The expected final product is obtained in the form of a light brown solid.

Melting point: 120.0-121.0. MH+=411.22.

### Example 35

### {4-[4-(3,5-di-tert-butyl-2-methoxyphenyl)-1,3-thiazol-2-yl]tetrahydro-2H-pyran-4-yl}dimethylamine hydrochloride

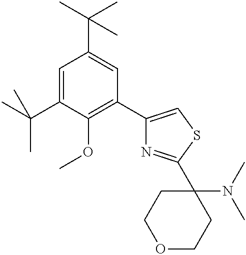

The experimental protocol used is the same as the one described for examples 1 and 2 with 3,5-di-tert-butyl-2-methoxyacetophenone replacing 3,5-di-tert-butyl-4-hydroxy acetophenone. The expected final product is obtained in the form of a light yellow solid.

Melting point: 199.4-200.7° C. MH+=431.22.

### Pharmacological Study of the Products of the Invention

The affinity of the compounds of the present invention for the different cannabinoid receptor subtypes was determined using procedures similar to those described hereinbelow for the human CB2 receptor.

### Study of the Affinity of the Compounds for Human Cannabinoid CB2 Receptors

The affinity of the compounds of the invention for human CB2 receptors was determined by measuring inhibition of [³H]-CP55940 binding to membrane preparations produced from transfected CHO-K1 cells.

CHO-K1 cells stably expressing human CB2 receptors were cultured in RPMI 1640 medium supplemented with 10% foetal calf serum, 2 mM glutamine, 100 U/ml penicillin, 0.1 mg/ml streptomycin and 0.5 mg/ml G418. The cells were collected using 0.5 mM EDTA and centrifuged at 500 g for 5 min at 4° C. The cell pellet was resuspended in phosphate buffered saline (PBS) and centrifuged at 500 g for 5 min at 4° C. The pellet was resuspended in 50 mM tris buffer pH 7.4 and centrifuged at 500 g for 5 min at 4° C. The cells were lysed by sonication and centrifuged at 39,000 g for 10 min at 4° C. The pellet was resuspended in 50 mM tris buffer pH 7.4 and centrifuged at 50,000 g for 10 min at 4° C. The membranes obtained in this final pellet were stored at −80° C.

Competitive inhibition of the binding of [³H]-CP55940 to CB2 receptors was determined in duplicate in 96-well polypropylene plates. Cell membranes (10 μg of protein/well) were incubated with [³H]-CP55940 (1 nM) for 60 min at 25° C. in 50 mM Tris-HCl buffer, pH 7.4, containing 0.1% bovine serum albumin (BSA), 5 mM MgCl₂, and 50 μg/ml of bacitracin.

Bound [³H]-CP55940 was separated from free [³H]-CP55940 by filtration through GF/C fibre glass filter plates (Unifilter, Packard) pre-impregnated with 0.1% polyethylenimine (P.E.I.), using a Filtermate 196 (Packard). The filters were washed with 50 mM Tris-HCl buffer, pH 7.4 at 0-4° C. and the radioactivity present was determined using a counter (Packard Top Count).

Specific binding was obtained by subtracting non-specific binding (determined in the presence of 0.1 μM WIN55212-2) from total binding. The data were analysed by computer-assisted nonlinear regression (MDL). For each test, the IC50 value was converted to a Ki value (inhibition constant) using the Cheng-Prusoff equation.

$$Thus , Ki = \frac{IC 50}{1 + [ L ] / Kd}$$

where [L] is the concentration of the radioligand used in the assay and Kd is the dissociation constant of the radioligand at equilibrium.

Any agonist or antagonist activity exhibited by the compounds of the present invention on CB2 receptors was determined by measuring cyclic AMP production by CHO-K1 cells transfected with the CB2 receptor.

### Determination of CB2 Receptor-Mediated Generation of Intracellular Cyclic AMP:

CHO-K1 cells expressing CB2 cannabinoid receptors were cultured in 384-well plates in RPMI 1640 medium supplemented with 10% foetal calf serum and 0.5 mg/ml G418. The cells were washed twice with 50 μl of RPMI medium containing 0.2% BSA and 0.5 mM 3-isobutyl-1-methylxanthine (IBMX).

To measure the agonist effect of a compound, the cells were incubated for 5 min at 37° C. in the presence of 0.5 mM IBMX, then cyclic AMP production was activated by adding 5 μM of Forskolin and inhibition was measured by adding the compound at concentrations ranging from 1 μM to 10 μM in duplicate followed by incubation at 37° C. for 20 min. The antagonist effect of a compound was measured by inhibiting the inhibition of cyclic AMP production mediated by WIN55212-2 in the presence of 5 μM Forskolin, at concentrations ranging from 1 μM to 10 μM, in the presence of the compound to be tested, at concentrations ranging from 1 nM to 10 μM, in duplicate, for 20 min at 37° C.

The reaction medium was eliminated and 80 μl of lysis buffer were added. Intracellular cyclic AMP levels were measured in a competitive assay using fluorescent cyclic AMP (CatchPoint, Molecular Devices).

### Results of the Pharmacological Studies:

a) All the compounds of examples 1 to 35 are selective for the CB2 receptor relative to the CB1 receptor. Selective is understood in the present invention to mean that the compounds have higher affinity for the CB2 receptor than for the CB1 receptor.
b) Generally, the affinity of all the compounds of examples 1 to 35 for the CB2 receptor is less than 10,000 nM.

The affinity of examples 5, 14, 28, 29 and 33 is between 1000 nM and 500 nM.

The affinity of examples 7, 8, 11, 13, 16, 17, 19, 21, 23, 25 and 32 is between 500 nM and 100 nM.

The affinity of examples 2, 4, 6, 15, 18 and 20 is less than or equal to 100 nM.

1. Compound having the general formula (I) 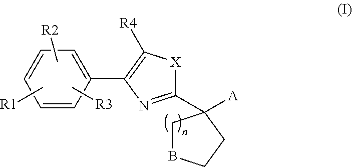
in a racemic form, an enantiomeric form or any combinations thereof, wherein:
A represents an —NR5R6, —CR5R6R7 or —OR5 group, where R5, R6, R7 represent independently a hydrogen atom, an alkyl or cycloalkyl group;
or alternatively A represents —NR5R6, where R5 and R6 together with the nitrogen atom to which they are attached form a heterocycloalkyl;
or alternatively A represents —CR5R6R7, where R5, R6 and R7 together with the carbon atom to which they are attached form a cycloalkyl;
n represents an integer between 1 and 3 inclusive;
B represents an oxygen atom, a sulphur atom, a methylene group or an —NR9 group;
X represents an oxygen or sulphur atom;
R4 represents a hydrogen atom or an alkyl group;
R1, R2, R3 represent independently a hydrogen atom, a halogen atom, an alkyl group, —CH₂R11, —SR11, haloalkyl, —N(R9)₂, —OR10;
R9 represents a hydrogen atom or an alkyl group;
R10 represents a hydrogen atom, an alkyl or aryl group optionally substituted by one or more identical or independently different groups selected from halo, nitro, cyano, or alkoxy;
R11 represents an aryl group;
or a pharmaceutically acceptable salt thereof;
it being understood that when A represents —NH₂, B does not represent a methylene group.

2. Compound according to claim 1 characterised in that R1, R2, R3 represent independently a hydrogen atom, a halogen atom, an alkyl group, —CH₂R11, —SR11, haloalkyl, —N(R9)₂₎, —OR10; R10 represents a hydrogen atom, an alkyl or phenyl group optionally substituted by one or more identical or independently different groups selected from halo, nitro, cyano, or alkoxy; and R11 represents a phenyl group.

3. Compound according to any one of claim 1 or 2 characterised in that R1, R2, R3 represent independently a hydrogen atom, a halogen atom, an alkyl group, —CH₂R11, —SR11, haloalkyl, —N(R9)₂, —OR10; R10 represents a hydrogen atom, an alkyl or phenyl group optionally substituted by one or more identical or independently different groups selected from halo, nitro, cyano or methoxy; and R11 represents a phenyl group.

4. Compound according to claim 1 characterised in that X represents the sulphur atom.

5. Compound according to claim 1 characterised in that X represents the oxygen atom.

6. Compound according to claim 1 characterised in that R1, R2, R3 represent independently a hydrogen atom, a halogen atom, an alkyl group or —OR10.

7. Compound according to claim 1 characterised in that R1, R2, R3 represent independently an alkyl group or —OR10, it being understood that R10 represents a hydrogen atom or an alkyl group.

8. Compound according to claim 1 characterised in that A represents an —NR5R6 group where R5 and R6 represent independently a hydrogen atom or an alkyl group.

9. Compound according to claim 1 characterised in that A represents —NR5R6 where R5 and R6 together with the nitrogen atom to which they are attached form a heterocycloalkyl.

10. Compound according to claim 1 characterised in that B represents an oxygen or sulphur atom.

11. Compound according to claim 1 characterised in that A represents independently an —NR5R6 group, where R5 and R6 represent independently a hydrogen atom, an alkyl or cycloalkyl group;
or alternatively A represents —NR5R6, where R5 and R6 together with the nitrogen atom to which they are attached form a heterocycloalkyl;
n represents an integer between 1 and 3 inclusive;
B represents an oxygen atom, a sulphur atom, a methylene group or an —NR9 group;
X represents an oxygen or sulphur atom;
R4 represents a hydrogen atom or an alkyl group;
R1, R2, R3 represent independently a hydrogen atom, a halogen atom, an alkyl group, —OR10;
R9 represents a hydrogen atom or an alkyl group;
R10 represents a hydrogen atom, an alkyl or aryl group optionally substituted by one or more identical or independently different groups selected from halo, nitro, cyano, or alkoxy;
or a pharmaceutically acceptable salt thereof;
it being understood that when A represents —NH₂, B does not represent a methylene group.

12. Compound according to claim 1 characterised in that A represents an —NR5R6 group, where R5 and R6 represent independently a hydrogen atom, an alkyl or cycloalkyl group;
or alternatively A represents —NR5R6, where R5 and R6 together with the nitrogen atom to which they are attached form a heterocycloalkyl;
n represents an integer between 1 and 3 inclusive;
B represents an oxygen or sulphur atom;
X represents an oxygen or sulphur atom;
R4 represents a hydrogen atom or an alkyl group;
R1, R2, R3 represent independently a hydrogen atom, a halogen atom, an alkyl group, —CH₂R11, —SR11, haloalkyl, —N(R9)₂, —OR10;
R10 represents a hydrogen atom, an alkyl or aryl group optionally substituted by one or more identical or independently different groups selected from halo, nitro, cyano or alkoxy;
R11 represents an aryl group;
or a pharmaceutically acceptable salt thereof.

13. Compound according to claim 1 characterised in that A represents an —NR5R6 group, where R5 and R6 represent independently a hydrogen atom, an alkyl or cycloalkyl group;
or alternatively A represents —NR5R6, where R5 and R6 together with the nitrogen atom to which they are attached form a heterocycloalkyl;
n represents an integer between 1 and 3 inclusive;
B represents an oxygen or sulphur atom;
X represents an oxygen or sulphur atom;
R4 represents a hydrogen atom or an alkyl group;
R1, R2, R3 represent independently a hydrogen atom, a halogen atom, an alkyl group or —OR10;
R10 represents a hydrogen atom, an alkyl or aryl group optionally substituted by one or more identical or independently different groups selected from halo, nitro, cyano or alkoxy;
or a pharmaceutically acceptable salt thereof.

14. Compound according to claim 1 characterised in that said compound is selected from the following compounds or a salt thereof:
{4-[4-(3,5-di-tert-butyl-4-methoxyphenyl)-1,3-thiazol-2-yl]tetrahydro-2H-pyran-4-yl}amine
{4-[4-(3,5-di-tert-butyl-4-methoxyphenyl)-1,3-thiazol-2-yl]tetrahydro-2H-pyran-4-yl}dimethylamine hydrochloride
4-[2-(4-aminotetrahydro-2H-pyran-4-yl)-1,3-thiazol-4-yl]-2,6-di-tert-butylphenol
2,6-di-tert-butyl-4-{2-[4-(dimethylamino)tetrahydro-2H-pyran-4-yl]-1,3-thiazol-4-yl}phenol hydrochloride
4-[2-(4-aminotetrahydro-2H-thiopyran-4-yl)-1,3-thiazol-4-yl]-2,6-di-tert-butyl phenol
2,6-di-tert-butyl-4-{2-[4-(dimethylamino)tetrahydro-2H-thiopyran-4-yl]-1,3-thiazol-4-yl}phenol hydrochloride
2,6-di-tert-butyl-4-{2-[3-(dimethylamino)tetrahydrofuran-3-yl]-1,3-thiazol-4-yl}phenol hydrochloride
{4-[4-(4-tert-butylphenyl)-1,3-thiazol-2-yl]tetrahydro-2H-pyran-4-yl}dimethylamine hydrochloride
(4-{-4-[3,5-bis(trifluoromethyl)phenyl]-1,3-thiazol-2-yl}tetrahydro-2H-pyran-4-yl)dimethylamine hydrochloride
{4-[4-(3,5-di-tert-butylphenyl)-1,3-thiazol-2-yl]tetrahydro-2H-pyran-4-yl}amine
{4-[4-(3,5-di-tert-butylphenyl)-1,3-thiazol-2-yl]tetrahydro-2H-pyran-4-yl}dimethylamine hydrochloride
{4-[4-(4-methoxy-3,5-dimethylphenyl)-1,3-thiazol-2-yl]tetrahydro-2H-pyran-4-yl}dimethylamine hydrochloride
{4-[4-(3-tert-butyl-5-chloro-4-methoxyphenyl)-1,3-thiazol-2-yl]tetrahydro-2H-pyran-4-yl}dimethylamine hydrochloride
{4-[4-(3-tert-butyl-4-methoxyphenyl)-1,3-thiazol-2-yl]tetrahydro-2H-pyran-4-yl}dimethylamine hydrochloride
2,6-di-tert-butyl-4-{2-[4-(dimethylamino)tetrahydro-2H-pyran-4-yl]-1,3-oxazol-4-yl}phenol hydrochloride
N,N-dimethyl-4-[4-(4-phenoxyphenyl)-1,3-thiazol-2-yl]tetrahydro-2H-pyran-4-amine hydrochloride
2,6-di-tert-butyl-4-{2-[4-(ethylamino)tetrahydro-2H-pyran-4-yl]-1,3-thiazol-4-yl}phenol
2,6-di-tert-butyl-4-{2-[4-(diethylamino)tetrahydro-2H-pyran-4-yl]-1,3-thiazol-4-yl}phenol hydrochloride
2,6-di-tert-butyl-4-{2-[4-(methylamino)tetrahydro-2H-pyran-4-yl]-1,3-thiazol-4-yl}phenol hydrochloride
2,6-di-tert-butyl-4-[2-(4-piperidin-1-yltetrahydro-2H-pyran-4-yl)-1,3-thiazol-4-yl]phenol hydrochloride
2,6-di-tert-butyl-4-[2-(4-morpholin-4-yltetrahydro-2H-pyran-4-yl)-1,3-thiazol-4-yl]phenol
2,6-di-tert-butyl-4-{2-[4-(4-methylpiperazin-1-yl)tetrahydro-2H-pyran-4-yl]-1,3-thiazol-4-yl}phenol hydrochloride
2,6-di-tert-butyl-4-{2-[4-(dimethylamino)-1-methylpiperidin-4-yl]-1,3-thiazol-4-yl}phenol hydrochloride
2,6-di-tert-butyl-4-{2-[4-(dimethylamino)piperidin-4-yl]-1,3-thiazol-4-yl}phenol trifluoroacetate
{4-[4-(3,5-di-tert-butyl-4-methoxyphenyl)-5-methyl-1,3-thiazol-2-yl]tetrahydro-2H-pyran-4-yl}dimethylamine hydrochloride
2,6-di-tert-butyl-4-[2-(1-methoxycyclopentyl)-1,3-thiazol-4-yl]phenol
2,6-di-tert-butyl-4-[2-(1-ethylcyclopentyl)-1,3-thiazol-4-yl]phenol
(4-{4-[4-(diethylamino)phenyl]-1,3-thiazol-2-yl}tetrahydro-2H-pyran-4-yl) dimethylamine hydrochloride
{4-[4-(4-benzylphenyl)-1,3-thiazol-2-yl]tetrahydro-2H-pyran-4-yl}dimethylamine hydrochloride
2-chloro-6-(4-{2-[4-(dimethylamino)tetrahydro-2H-pyran-4-yl]-1,3-thiazol-4-yl}phenoxy)benzonitrile hydrochloride
(4-{-4-[2-chloro-4-(4-chlorophenoxy)phenyl]-1,3-thiazol-2-yl}tetrahydro-2H-pyran-4-yl)dimethylamine hydrochloride
N,N-dimethyl-4-{4-[4-(4-nitrophenoxy)phenyl]-1,3-thiazol-2-yl}tetrahydro-2H-pyran-4-amine hydrochloride
N,N-dimethyl-4-{4-[4-(phenylthio)phenyl]-1,3-thiazol-2-yl}tetrahydro-2H-pyran-4-amine hydrochloride
(4-{4-[4-(4-methoxyphenoxy)phenyl]-1,3-thiazol-2-yl}tetrahydro-2H-pyran-4-yl)dimethylamine hydrochloride
{4-[4-(3,5-di-tert-butyl-2-methoxyphenyl)-1,3-thiazol-2-yl]tetrahydro-2H-pyran-4-yl}dimethylamine hydrochloride.

15. Compound according to claim 1 characterised in that said compound is selected from the following compounds or a salt thereof:
{4-[4-(3,5-di-tert-butyl-4-methoxyphenyl)-1,3-thiazol-2-yl]tetrahydro-2H-pyran-4-yl}dimethylamine hydrochloride
2,6-di-tert-butyl-4-{2-[4-(dimethylamino)tetrahydro-2H-pyran-4-yl]-1,3-thiazol-4-yl}phenol hydrochloride
2,6-di-tert-butyl-4-{2-[4-(dimethylamino)tetrahydro-2H-thiopyran-4-yl]-1,3-thiazol-4-yl}phenol hydrochloride
2,6-di-tert-butyl-4-{2-[4-(dimethylamino)tetrahydro-2H-pyran-4-yl]-1,3-oxazol-4-yl}phenol hydrochloride
2,6-di-tert-butyl-4-{2-[4-(diethylamino)tetrahydro-2H-pyran-4-yl]-1,3-thiazol-4-yl}phenol hydrochloride
2,6-di-tert-butyl-4-[2-(4-piperidin-1-yltetrahydro-2H-pyran-4-yl)-1,3-thiazol-4-yl]phenol hydrochloride.

16. Process for preparing a compound of general formula (I) according to claim 1 characterised in that an α-halogenoketone of general formula (4) 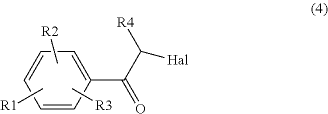
wherein R1, R2, R3 and R4 are as hereinbefore defined and Hal represents a halogen atom,
is reacted either with a compound of general formula (30) 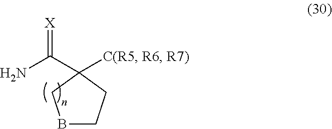
wherein X, B, R5, R6, R7 and n are as hereinbefore defined, to produce a compound having the general formula (32) i.e. a compound of general formula (I) where A represents a —CR5R6R7 group 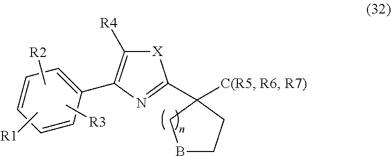
wherein R1, R2, R3, R4, X, B, n, R5, R6 and R7 are as defined in claim 1;
or is reacted with a compound of general formula (40) 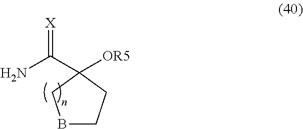
wherein X, B, R5 and n are as hereinbefore defined, to produce the compound having the general formula (42) i.e. a compound of general formula (I) where A represents an —OR5 group 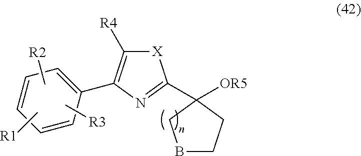
wherein R1, R2, R3, R4, X, B, n and R5 are as defined in claim 1;
or is reacted with a compound of general formula (6) 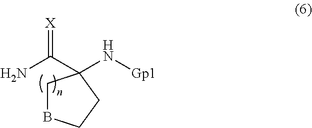
wherein X, B and n are as hereinbefore defined and Gp1 represents a protecting group, to produce the compound having the general formula (8) 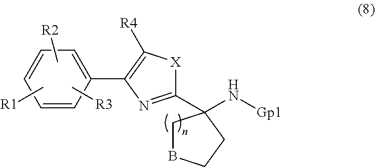
wherein R1, R2, R3, R4, X, B, n and Gp1 are as hereinbefore defined; compound of general formula (8) of which the protecting group is cleaved to produce the compound of general formula (9), i.e. a compound of general formula (I) wherein A represents an —NR5R6 group, R5 and R6 representing a hydrogen atom 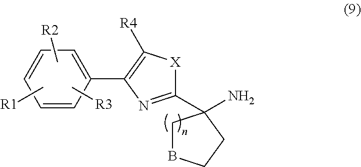
compound of general formula (I) wherein R1, R2, R3, R4, X, B, and n are as hereinbefore defined and A represents an —NR5R6 group, R5 and R6 representing a hydrogen atom, which is used as an intermediate for the synthesis of the compound of general formula (14) i.e. a compound of general formula (I) wherein A represents an —NR5R6 group, and R5 and/or R6 do not represent a hydrogen atom 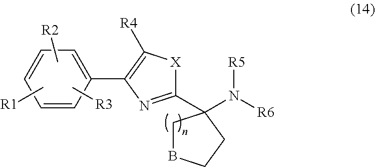
wherein R1, R2, R3, R4, X, B, n, R5 and R6 are as hereinbefore defined and R5 and/or R6 do not represent a hydrogen atom.

17. A compound of general formula (I) according to any one of claims 1 to 15, or a pharmaceutically acceptable salt thereof, as a drug.

18. Pharmaceutical composition containing as an active substance a compound of general formula (I) according to any one of claims 1 to 15, or a pharmaceutically acceptable salt of such a compound, with at least one pharmaceutically acceptable excipient.

19. Use of at least one compound of general formula (I) according to any one of claims 1 to 15, or a pharmaceutically acceptable salt thereof, to prepare a drug intended from the treatment or prevention of a disease or disorder selected from the following diseases or disorders: disorders of cell proliferation such as cancer, immune disorders and autoimmune diseases, allergic diseases, inflammation, pain, eye disorders, lung conditions, osteoporosis, gastrointestinal disorders, neurodegenerative diseases, cardiovascular diseases.

20. Use according to claim 19, characterised in that the drug prepared is intended for the treatment or prevention of cancer.

21. Use according to claim 20, characterised in that the cancer to be treated or prevented is selected from cancer of the colon, rectum, stomach, lung, pancreas, kidney, testicle, breast, uterus, ovary, prostate, skin, bone, spinal cord, neck, tongue or head, as well as sarcomas, carcinomas, fibroadenomas, neuroblastomas, leukaemias and melanomas.
